(19) United States
(12) Patent Application Publication
(10) Pub. No.: US 2019/0083589 A1
(43) Pub. Date: Mar. 21, 2019

(54) METHODS FOR PRODUCING PEPTIDES AND USES THEREOF

(71) Applicant: AMIDEBIO, LLC, Boulder, CO (US)

(72) Inventors: Michael H.B. STOWELL, Boulder, CO (US); Stephen EISENBERG, Boulder, CO (US); Yanyu PENG, Superior, CO (US); Mikhail PLAM, Boulder, CO (US)

(21) Appl. No.: 16/084,934

(22) PCT Filed: Mar. 16, 2017
(86) PCT No.: PCT/US17/22806

§ 371 Date: Sep. 13, 2018

Related U.S. Application Data
(60) Provisional application No. 62/309,381, filed on Mar. 16, 2016.

(51) Int. Cl. A61K 38/56; C07K 7/06; C07K 14/81; C07K 14/415; C12N 15/62; A61P 3/10; A61P 37/06; C07K 14/47; C07K 14/62; A61K 38/26; A61K 9/00

(52) U.S. Cl. CPC A61K 38/56; C07K 7/06; C07K 14/81; C07K 14/415; C12N 15/62; A61K 9/0053; A61P 37/06; C07K 14/4713; C07K 14/62; A61K 38/26; A61P 3/10

(57) ABSTRACT

The present disclosure relates to processes for the production of peptides and methods of using peptides produced accordingly. Peptides produced according to the present disclosure may be administered for the treatment of autoimmune and/or inflammatory diseases and conditions.

### CROSS-REFERENCE TO RELATED APPLICATIONS

This application is related to U.S. provisional patent application No. 62/309,381, filed Mar. 16, 2016, which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Feb. 27, 2017, is named 39538-710_601_SL.txt and is 4,077 bytes in size.

### BACKGROUND OF THE INVENTION

Peptides have various uses in basic research and clinical practice, for example functioning as mediators in various biological pathways and possessing intrinsic biological properties. Peptides can exhibit specific binding, having high specificity for their target interaction partners and low specificity for non-target molecules. Peptides may also show low accumulation in tissues over time, thus reducing side effects when administered. Moreover, peptides can be broken down in vivo into their constituent amino acids, thus reducing the risk of complications due to toxic metabolic intermediates.

The life sciences peptide market can be broadly grouped into five categories—cytokines, enzymes, hormones, antibodies, and vaccines. These categories can be further subdivided into vaccines, monoclonal and polyclonal antibodies, recombinant hormones and proteins, gene therapy, cell therapy, antisense, interferons, interleukins, growth factors, and others.

While advances in the field of peptide science have led to commercial growth, several factors may limit the widespread use of peptide therapeutics. For example, peptides may be associated with delivery and stability problems compared to traditional small molecule therapeutics. Attempts to address these problems have involved oral, nasal, and pulmonary routes of administration. However, these alternatives may require higher doses of the peptides or yield unfavorable pharmacokinetic profiles. Additionally, the production costs of peptide therapeutics may exceed that of small molecule therapeutics.

### SUMMARY OF THE INVENTION

In view of the forgoing, improved means of peptide production and purification are desirable, where the improvement(s) may be in production efficiency, cost efficiency, and/or product quality. Accordingly, the present invention is directed to methods for the production of peptides and uses thereof.

In an aspect, a pharmaceutical composition comprises a soybean Bowman-Birk inhibitor (BBI) protein having at least 80% sequence identity to SEQ ID NO: 1 (DDESSKPCCDQCACTKSNPPQCRCSDMRLNSCHSACKSCICALSYPAQCFCVDI TDFCYEPCKPSEDDKEN) or a fragment thereof, and a pharmaceutically acceptable diluent, wherein the soybean BBI protein comprises a methionine sulfoxide, a valine, a leucine or isoleucine at amino acid 27. In some embodiments, the soybean BBI protein has at least 85%, 90%, 95%, or 98% sequence identity to SEQ ID NO: 1. In some embodiments, the soybean BBI protein has an amino acid sequence of SEQ ID NO: 1.

In some embodiments, the pharmaceutical composition further comprises a peptide. In some embodiments, the peptide is an insulin peptide, analogue or fragment thereof; a glucagon peptide, analogue or fragment thereof; and a glucagon-like peptide-1 (GLP-1) peptide, analogue or fragment thereof. In some embodiments, the pharmaceutical composition is formulated for oral administration.

In an aspect, a method for treating an autoimmune disease comprises administering to a subject in need thereof any of the pharmaceutical compositions disclosed herein. In some embodiments, the autoimmune disease is selected from the group consisting of: type I diabetes, Stevens-Johnson Syndrome, Guillain-Barre Syndrome, anti-aquaporin 4 antibody positive neuromyelitis optica spectrum disorder, and bullous pemphigoid. In some embodiments, the autoimmune disease is type I diabetes. In some embodiments, the peptide is an insulin peptide, analogue or fragment thereof. In some embodiments, the pharmaceutical composition is administered orally.

In an aspect, a method for producing a target peptide comprises expressing a heterologous fusion peptide in a genetically modified cell, the heterologous fusion peptide comprising an expression tag, a cleavage tag, and the target peptide, wherein the expression tag comprises an amino acid sequence having at least 80% sequence identity to SEQ ID NO: 2 (MKAIFVLKGSLDRDPEFPSDKPHHKKHHKKHHSSGSLE) or a fragment thereof, and wherein the cleavage tag comprises a Trp (W) amino acid; and cleaving the heterologous fusion peptide to release the target peptide from the heterologous fusion peptide, thereby producing the target peptide.

In some embodiments, the target peptide is selected from the group consisting of: a hormone peptide, a protease inhibitor protein, and a peptide toxin. In some embodiments, the target peptide is selected from the group consisting of: insulin, glucagon, glucagon-like peptide 1 (GLP-1), parathyroid hormone 1-34 (PTH-34), a single-chain relaxin-1, a single-chain relaxin-2, a single-chain relaxin-3, insulin-like peptide 3, insulin-like peptide 4, insulin-like peptide 5, insulin-like peptide 6, soybean trypsin inhibitor (STI) protein, soybean Bowman-Birk inhibitor (BBI) protein, eglin C protein, Mambalgin-1, Hg1 toxin, and Stichodactyla toxin (ShK).

In some embodiments, the target peptide is a soybean BBI protein having at least 80%, 85%, 90%, 95%, 98%, or 100% sequence identity to SEQ ID NO: 1 (DDESSKPCCDQCACTKSNPPQCRCSDMRLNSCHSACKSCICALSYPAQCFCVDITDFCY EPCKPSEDDKEN) or a fragment thereof.

In some embodiments, the soybean BBI protein comprises an oxidized amino acid. In some embodiments, the soybean BBI protein comprises a methionine sulfoxide at amino acid 27. In some embodiments, the soybean BBI protein comprises a valine, leucine or isoleucine at amino acid 27. In some embodiments, the target peptide is at least 95% pure. In some embodiments, the target peptide is at least 99% pure.

In some embodiments, the expression tag further comprises an affinity tag. In some embodiments, the affinity tag comprises at least six amino acids having charged side chains. In some embodiments, the method further comprises binding the heterologous fusion peptide to an affinity material via the affinity tag. In some embodiments, subsequent to binding the heterologous fusion peptide to the affinity material, the method further comprises washing the affinity material to remove unbound material. In some embodiments, cleaving the heterologous fusion peptide in (b) occurs while the heterologous fusion peptide is bound to the affinity material via the affinity tag.

In some embodiments, the target peptide possesses a tertiary structure substantially the same as the corresponding native target peptide after cleaving. In some embodiments, subsequent to binding the heterologous fusion peptide to the affinity material, the method further comprises subjecting the heterologous fusion peptide to conditions sufficient to fold the target peptide.

In some embodiments, the heterologous fusion peptide further comprises an inclusion-body directing peptide. In some embodiments, the inclusion-body directing peptide is selected from the group consisting of: a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, and an inclusion-body directing functional homolog of BRCA2. In some embodiments, prior to cleaving the heterologous fusion peptide, the method further comprises removing inclusion bodies containing the fusion peptide from the genetically modified cell and solubilizing the fusion peptide in the inclusion bodies.

In some embodiments, the cleaving of (b) is performed with an agent selected from the group consisting of: NBS, NCS, and Pd(H2O)4.

In some embodiments, the heterologous fusion peptide is secreted from the genetically modified cell after it is expressed. In some embodiments, the method further comprises lysing the genetically modified cell after the heterologous fusion peptide is expressed.

In some embodiments, the genetically modified cell is a bacterial cell. In some embodiments, the bacterial cell is an Escherichia coli cell. In some embodiments, the genetically modified cell is a yeast cell. In some embodiments, the heterologous fusion peptide further comprises a secretion peptide for use in the yeast cell.

In an aspect, a vector comprises a first nucleotide sequence encoding an expression tag; a second nucleotide sequence encoding a cleavage tag; and a third nucleotide sequence encoding a target peptide; wherein the first, second, and third nucleotide sequences are arranged in operable combination, wherein the expression tag comprises an amino acid sequence having at least 80% sequence identity to SEQ ID NO: 2 (MKAIFVLKGSLDRDPEFPSDKPHHKKHHKKHHSSGSLE) or a fragment thereof, and wherein the cleavage tag comprises a Trp (W) amino acid.

In some embodiments, the target peptide is selected from the group consisting of: a hormone peptide, a protease inhibitor, and a peptide toxin. In some embodiments, the target peptide is selected from the group consisting of: insulin, glucagon, glucagon-like peptide 1 (GLP-1), parathyroid hormone 1-34 (PTH-34), a single-chain relaxin-1, a single-chain relaxin-2, a single-chain relaxin-3, insulin-like peptide 3, insulin-like peptide 4, insulin-like peptide 5, insulin-like peptide 6, soybean trypsin inhibitor (STI) protein, soybean Bowman-Birk inhibitor (BBI) protein, eglin C protein, Mambalgin-1, Hg1 toxin, and Stichodactyla toxin (ShK).

In some embodiments, the target peptide is a soybean BBI protein having at least 80%, 85%, 90%, 95%, or 98% sequence identity to SEQ ID NO: 1 (DDESSKPCCDQCACTKSNPPQCRCSDMRLNSCHSACKSCICALSYPAQCFCVDITDFCY EPCKPSEDDKEN) or a fragment thereof. In some embodiments, the soybean BBI protein has an amino acid sequence of SEQ ID NO: 1.

In some embodiments, the expression tag further comprises an affinity tag. In some embodiments, the affinity tag comprises at least six amino acids having charged side chains. In some embodiments, the vector further comprises a nucleotide sequence encoding an inclusion-body directing peptide. In some embodiments, the inclusion-body directing peptide is selected from the group consisting of: a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, and an inclusion-body directing functional homolog of BRCA2. In some embodiments, the vector further comprises a nucleotide promoter sequence which is active in a bacteria cell or a yeast cell.

### Incorporatin by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 presents the chemical structures of a variety of unnatural amino acids that have been incorporated into peptides and proteins by cell systems through genetic modification of the cell systems.

FIG. 2 illustrates activation of transcription in a commercially available pBAD promoter via the addition of L-arabinose. Arabinose binds to AraC (“C” in the diagram) and causes the protein to release the 0₂ site and bind the 1₂ site which is adjacent to the Iᵢ site. This releases the DNA loop and allows transcription to begin. A second level of control is present in the cAMP activator protein (CAP)-cAMP complex, which binds to the DNA and stimulates binding of AraC to I₁ and I₂. Basal expression levels can be repressed by introducing glucose to the growth medium, which lowers cAMP levels and in turn decreases the binding of CAP, thus decreasing transcriptional activation.

FIG. 3 illustrates an immobilized Ni-NTA resin binding to a 6× His tag (SEQ ID NO: 4) on a protein.

FIG. 4 illustrates one possible mechanism for the selective cleavage of tryptophan peptide bonds with NBS (N-bromosuccinimide). According to the mechanism, the active bromide ion halogenates the indole ring of the tryptophan residue followed by a spontaneous dehalogenation through a series of hydrolysis reactions. These reactions lead to the formation of an oxindole derivative which promotes the cleavage reaction. Z-Trp-Y is cleaved at the carboxy terminus of the Trp residue to yield a modified Z-Trp and a free amino group on Y (i.e., H₂N—Y).

FIG. 5 shows results of mass spec analysis of BBI protein.

FIG. 6 shows results from trypsin inhibition assays using BBI protein having a methionine sulfoxide at amino acid 27.

FIG. 7 shows results from chymotrypsin inhibition assays using BBI protein having a methionine sulfoxide at amino acid 27.

### DETAILED DESCRIPTION OF THE INVENTION

The present invention provides methods for producing fusion peptides that can be purified and cleaved to produce desired target peptides and compositions comprising the target peptides produced according to the methods described herein. Methods for producing fusion peptides include induction of peptide or protein expression, inclusion body isolation, affinity column purification, and chemical cleavage. Also disclosed herein are expression vectors useful to produce target peptides. In some aspects, by combining molecular expression technologies that employ genetically-malleable microorganisms such as E. coli cells or yeast cells to synthesize a peptide of interest with post-expression isolation and modification, a desired target peptide can be produced rapidly and efficiently. Fusion peptides, such as heterologous fusion peptides, expressed using the methods described herein can be purified using affinity separation and cleaved with a chemical reagent to release a target peptide.

Peptides play various roles in human physiology, functioning, for example, as hormones, neurotransmitters, growth factors, and ion channel ligands. The terms “peptide,” “polypeptide,” and “protein” are used interchangeably herein to refer to a polymer of two or more amino acid residues joined by peptide bonds. This term does not connote a specific length of polymer amino acids, nor is it intended to imply or distinguish whether the peptide is produced using recombinant techniques, chemical or enzymatic synthesis, or is naturally occurring. The terms apply to naturally occurring amino acid polymers as well as amino acid polymers comprising one or more modified amino acids. In some cases, the polymer may be interrupted by non-amino acids. The terms include amino acid chains of any length, including full length proteins, and proteins with or without secondary and/or tertiary structure. The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

Peptides, including 1) synthetically and recombinantly produced peptides that mimic the function and/or properties of naturally occurring peptides and 2) engineered peptides that possess alternative biological properties compared to a naturally occurring peptide (e.g., antagonism or agonism of a cellular receptor), are being investigated as therapeutic molecules (e.g., peptide therapeutics). Peptide therapeutics include synthetic peptide hormones and neurotransmitters administered, for example, to treat homeostatic imbalance, such as glucoregulatory hormones administered to treat blood-glucose homeostatic imbalance. Peptide therapeutics may, in some cases, comprise peptide toxins administered to treat various diseases and disorders. Peptide toxins, for example those targeting ion channels, may be administered to treat various disorders associated with irregular ion channel activity, including immune disorders. Peptide therapeutics may also comprise enzyme inhibitors that can be administered to treat various medical conditions, for example conditions associated with irregular enzyme activity including autoimmune disorders and conditions.

### I. Peptide Therapeutics

Peptide and protein hormones may be involved in the endocrine system. Peptide hormones can interact with different cell types through cell surface and intracellular receptors to regulate various aspects physiology, including homeostasis (e.g., glucose homeostasis and calcium homeostasis) and immune system regulation. Natural peptide hormones can be produced in various organs and tissues, including the pituitary gland (e.g., prolactin, adrenocorticotropic hormone, and growth hormone); the heart (e.g., atrial-natriuretic peptide or atrial natriuretic factor); the pancreas (e.g., glucagon, insulin, and somatostatin); the gastrointestinal tract (e.g., cholecystokinin, gastrin, and glucagon-like peptide-1); the parathyroid (e.g., parathyroid hormone); and adipose tissue stores (e.g., leptin). Some peptide hormones function as neurotransmitters (e.g., neuropeptides). Binding of a peptide hormone to a receptor (e.g., a cell surface receptor or an intracellular receptor) can trigger signal transduction resulting in cellular responses.

Irregular release or misregulation of peptide hormones can result in disease conditions, including, but not limited to, diabetes mellitus, thyroid disease and obesity. In some cases where there is an insufficient amount of peptide hormone produced and/or released, synthetically or recombinantly produced peptide hormones may be administered to alleviate symptoms associated with the insufficient amount of endogenous peptide hormone.

For example, blood glucose levels are primarily regulated by the glucoregulatory hormones, such as insulin, glucagon, amylin, and incretins (e.g., glucagon-like peptide-1, GLP-1). Glucoregulatory hormones function to maintain circulating glucose concentrations within a desired range. Low blood glucose levels can stimulate the release of glucagon by alpha cells of the pancreas. Liver cells, in response to glucagon, convert glycogen into glucose in a process referred to as glycolysis. The glucose is released into the bloodstream, thereby increasing blood glucose levels. However, when blood glucose levels rise, whether as a result of glycogen conversion or from digestion of food, insulin is released from the pancreas. Insulin stimulates liver cells to convert glucose into glycogen in a process referred to as glycogenesis, thereby decreasing blood glucose levels. Together, glucagon and insulin function in a feedback system to maintain blood glucose levels at a stable level. Amylin, a peptide co-secreted with insulin from the pancreas, plays a role in blood glucose regulation by slowing gastric emptying and inhibiting digestive secretion. Incretins, which includes glucagon-like peptide-1 (GLP-1), are a group of metabolic hormones that stimulate a decrease in blood glucose levels. Glucagon-like peptide-1 (GLP-1) is secreted primarily from the intestinal L-cells in response to food and modulates nutrient homeostasis via actions exerted in multiple tissues and cell types.

Irregular release or misregulation of any of the above mentioned peptide hormones may result in various medical conditions, including hyperglycemia and hypoglycemia. Chronic irregularities in the levels of these hormones may result in conditions including diabetes mellitus type 1, also referred to as type 1 diabetes. In some cases, glucoregulatory peptides, such as those produced synthetically or recombinantly, may be administered to treat conditions associated with misregulation of glucoregulatory hormones. For example, insulin peptides, glucagon peptides, and/or glucagon-like peptide-1 (GLP-1) peptides, analogues or fragments thereof can be administered to treat conditions associated with the irregular release or misregulation of peptide hormones.

As a further example, calcium homeostasis is primarily regulated by the peptide hormone parathyroid hormone (PTH) and a metabolite of vitamin D. Parathyroid hormone is secreted by the chief cells of the parathyroid glands as a polypeptide containing 84 amino acids, however, the 34 N-terminal amino acids (e.g., PTH 1-34) are sufficient to interact with the hormone-receptor. Calcium homeostasis, which refers to the regulation of calcium ions, is important as calcium has several main functions in the body. Calcium can serve as an intracellular signal or second messenger for various biological processes. As basal levels of intracellular calcium ion concentrations are relatively low, the entry of calcium ions from the endoplasmic reticulum or from extracellular fluid can cause rapid and readily reversible changes in the relative concentration of these ions in the cytosol. Calcium functions in various biological processes including muscle contraction and the release of hormones (e.g. insulin from the beta cells in the pancreatic islets) and neurotransmitters (e.g. acetylcholine from pre-synaptic terminals of nerves).

Voltage gated sodium ion channels in the cell membranes of nerves and muscle may be sensitive to the calcium ion concentration in the plasma. Relatively small decreases in the ionized calcium levels (hypocalcemia) may cause these channels to leak sodium into the nerve cells or axons, making them hyper-excitable (e.g., positive bathmotropic effect) and potentially causing spontaneous muscle spasms and paraesthesia of the extremities. When the ionized calcium levels rise above normal levels (e.g., hypercalcemia), more calcium may be bound to these sodium channels, resulting in a negative bathmotropic effect and potentially resulting in lethargy, muscle weakness, anorexia, and constipation.

Levels of PTH can become misregulated for various reasons, including thyroid removal or damage. Hypoparathyroidism, which refers to an abnormally low level of PTH, can result in abnormal levels of calcium and associated disorders. Synthetic or recombinantly produced PTH may be administered to treat hypoparathyroidism. In some cases, the 34 N-terminal amino acids (e.g., PTH 1-34) may be administered.

The relaxin family peptide hormones may also be involved in various biological processes. The relaxin family of peptide hormones includes three relaxin-like and four insulin-like peptides. The members of the relaxin-like peptide family include relaxin-1 (RLN1), relaxin-2 (RLN2), relaxin-3 (RLN3), insulin-like peptide 3 (INSL3), insulin-like peptide 4 (INSL4), insulin-like peptide 5 (INSL5), and insulin-like peptide 6 (INSL6). The relaxin family peptide hormones can be involved in reproductive functions, such as the relaxation of uterine musculature and of the pubic symphysis during labor; the progression of spermatogenesis; and possibly trophoblast development, testicular descent, and germ cell survival.

Disease conditions associated with the misregulated relaxin peptide hormone levels may be treated by administering synthetically or recombinantly produced relaxin peptide hormones, including relaxin-1 (RLN1), relaxin-2 (RLN2), relaxin-3 (RLN3), insulin-like peptide 3 (INSL3), insulin-like peptide 4 (INSL4), insulin-like peptide 5 (INSL5), and insulin-like peptide 6 (INSL6). In some cases, peptide analogues, such as single-chain relaxin-1, single-chain relaxin-2, and single-chain relaxin-3, having improved properties of thermal and/or plasma stability, decreased immunogenicity, or higher receptor binding affinity may be administered.

Peptide hormones including glucoregulatory hormones such as insulin, glucagon, and glucagon-like peptide-1 (GLP-1); PTH 1-34; relaxin family peptide hormones; or analogues thereof may be produced using methods described herein and administered as a peptide therapy. The term “analogue,” as used herein, refers to a protein that may be structurally and/or functionally similar to a native protein, for example a protein such as native glucoregulatory peptide (e.g., insulin, glucagon, and GLP-1) or a relaxin family peptide. An analogue may be structurally and/or functionally similar to a native protein, but is different in other various aspects, such as protein size (e.g., number of amino acids, molecular weight, diameter, etc.), amino acid sequence, amino acid composition, and tertiary structure.

### II. Peptide Toxins

Peptide toxin(s), herein also referred to as “toxin peptide(s),” refer to peptides, in some cases between about 20 and about 80 amino acids in length, that may be isolated from the venom of organisms including, but not limited to, invertebrates such as spiders, insects, and scorpions; fish such as stingrays; amphibians; and snakes. Such peptides may have various functions. In some cases, peptide toxin(s) can interact with ion channels which permit the exchange of small inorganic ions across membranes. Various ions (e.g., hydrogen, sodium, potassium, calcium, chloride, etc.) can move in and out of cells by passive diffusion through the plasma membrane. Ion channels situated in cell membranes may facilitate diffusion of ions into and out of cells. Ion channels can control the selective flux of ions across the membrane, thereby allowing for the formation of concentration gradients between the intracellular contents of the cell and the surrounding extracellular fluid. Ion channels are referred to as “gated” if they can be opened or closed. The basic types of gated ion channels include ligand gated channels, mechanically gated channels and voltage gated channels. Voltage gated channels can be found in neurons, muscle cells and non-excitable cells such as lymphocytes. Because ion concentrations are directly involved in the electrical activity of excitable cells (e.g., neurons), the functioning (or malfunctioning) of ion channels can influence the electrical properties and behavior of these cells.

A variety of disorders, broadly termed “channelopathies,” may be linked to ion channel insufficiencies or dysfunctions. Such disorders include autoimmune diseases such as multiple sclerosis, diabetes (e.g., type-1 diabetes), rheumatoid arthritis (RA), and psoriasis. In these disorders, specific autoreactive T cells—for instance myelin-specific T cells in MS patients—are believed to undergo repeated autoantigen stimulation during the course of the disease and differentiate into chronically activated memory cells that contribute to pathogenesis by migrating to inflamed tissues and secreting cytokines. Therapies that preferentially target chronically activated memory T cells may therefore be effective in treating autoimmune diseases. Non-limiting examples of peptide toxins that may be administered to treat channelopathies include apamin peptides, α-conopeptides, PnIA peptides, PnIB peptides, MII peptides, ShK toxin, BgK toxin, HmK toxin, AeKS toxin, AsK toxin, DTX1 toxin, Charybdotoxin (ChTx), Margatoxin (MgTx), Maurotoxin (MTx), OSK1 (α-KTx3.7), Kaliotoxin (KTX1), Agitoxin 2 (AgTx2), Pandinus imperator toxin (Pi2), Pandinus imperator toxin (Pi3), Noxiustoxin (NTX), Hg1 toxin, Hongotoxin (HgTx), BeKm-1 toxin, BmKTX, P01, BmKK6, Tc32, Tc1, BmTxI, BmTX3, IbTx, P05, ScyTx, TsK, HaTx1, ProTXI, PaTX2, Ptu1l, ωGVIA, ωMVIIA, SmIIIa, Anuoroctoxin (AnTx), Pi1, HsTx1, MTX (P12A, P20A), Pi4, Chlorotoxin, and Bm-12b.

In addition to autoimmune diseases and disorders, ion channels may be involved in energy homeostasis. Therefore, ion channel blockers may be administered to treat disorders associated with energy and homeostasis, such as obesity. Ion channels, such as Kv1.3, may play a role in regulating insulin-sensitivity in peripheral target organs such as the liver and muscle. It has been previously shown that genetic knockout of the Kv1.3 channel in mice enhanced the sensitivity of the liver and muscle to insulin. Peptide toxins that target ion channels, such as Kv1.3 blockers, may have use in the treatment of type-2 diabetes mellitus by enhancing the peripheral actions of insulin and thereby decreasing blood glucose levels. Kv1.3 may also be involved in regulating neurotransmitter release, heart rate, neuronal excitability, epithelial electrolyte transport, smooth muscle contraction and cell volume. Ion channel blockers that can block Kv1.3 include ShK toxin, a 35-residue polypeptide cross-linked by 3 disulfide bridges which can be found in the Caribbean sea anemone Stichodactyla helianthus, and Hg1 toxin, a scorpion Kunitz-type potassium channel toxin peptide.

In disease states, tissue acidosis may be a common pathologic change causing abnormal activation of acid-sensing ion channels (ASICs), which may contribute to inflammation, mitochondrial dysfunction, and other pathologic mechanisms (e.g., pain, stroke, and psychiatric conditions). Black Mamba toxic peptides (Mambalgins) have been found to inhibit ASICs and may have analgesic effects. Mambalgin-1, a peptide isolate of snake venom, selectively inhibits currents mediated by ASIC1 and ASIC1b homomers and heteromers can be administered to treat various diseases, including neurologic diseases.

Peptide toxins including ShK toxin, Hg1 toxin, and Mambalgins may be produced using methods described herein and administered as a peptide therapy.

### III. Protease Inhibitors as Disease-Modifying Therapeutics

Serine proteases, a sub-category of the protease family, are enzymes that cleave peptide bonds in proteins. Serine proteases may be involved in various physiological functions, including digestion, immune response, blood coagulation and reproduction. Serine proteases, such as trypsin and chymotrypsin, may also be involved in pathologic conditions and inflammation. Proteases and free radicals produced by macrophages and neutrophils, for example, may be associated with inflammation. Serine proteases have therefore been evaluated as therapeutic targets in inflammatory and autoimmune diseases, such as diabetes (e.g., type I diabetes), emphysema, Stevens-Johnson Syndrome (SJS), Guillain-Barre Syndrome (GBS), anti-aquaporin 4 antibody positive neuromyelitis optica spectrum disorder (NMOSD), and bullous pemphigoid. Inhibitors of chymotrypsin have been shown to be able to prevent the induction of superoxide anion radicals and hydrogen peroxide from stimulated human polymorphonuclear leukocytes and macrophage-like cells, thereby potentially reducing inflammation. Protease inhibitors may be administered for the treatment of autoimmune diseases characterized by chronic inflammation in a patient, such as rheumatoid arthritis, and for diseases that are characterized by chronic neuroinflammation and/or demyelination, such as multiple sclerosis (MS) and Guillain-Barre Syndrome (GBS).

Serine protease inhibitors, such as trypsin and chymotrypsin inhibitors, may potentially function as therapeutic agents in treating inflammatory and immune disorders. Protease inhibitors, such as serine protease inhibitors, can be administered to reduce, inhibit, suppress or prevent chronic inflammation and/or neuroinflammation in patients. Examples of serine protease inhibitors include soybean trypsin inhibitor; Bowman-Birk inhibitor (BBI) proteins from legumes (e.g., soybeans, adzuki beans, black beans, black-eyed peas, peas, lima beans, kidney beans, navy/white beans, pinto beans, chick peas, peanuts, lentils, etc); eglin C protease inhibitor from potatoes; bovine pancreas trypsin inhibitor (BPTI); serine leukocyte protease inhibitor (SLPI); and ovomucin (trypsin inhibitor found in egg white, e.g., chicken egg white, duck egg white, and turkey egg white).

The soybean Bowman-Birk protease inhibitor (BBI) (e.g., soybean BBI) can be resistant to temperature and acidic conditions. These characteristics may make it a good candidate for oral administration, with no major side effects. In addition, the therapeutic effect of BBI has been shown in inflammatory diseases and cancer. A soybean BBI protein can be produced using methods described herein and administered to treat diseases, such as inflammatory and/or autoimmune disease. A BBI inhibitor may have at least 60% sequence identity (e.g., at least 65%, 70%, 75%, 80%, 85%, 90%, 95% sequence identity or greater) to SEQ ID NO: 1 (DDESSKPCCDQCACTKSNPPQCRCSDMRLNSCHSACKSCICALSYPAQCFCVDITDFCY EPCKPSEDDKEN). As used herein, the terms “% sequence identity” and “% identical” with reference to a sequence, such as a polynucleotide sequence or a polypeptide sequence, refer to comparisons among polynucleotides and polypeptides when the sequences are optimally aligned over a comparison window. The portion of the polynucleotide or polypeptide sequence in the comparison window may comprise additions or deletions (e.g., gaps) as compared to the reference sequence (which does not comprise additions or deletions) for optimal alignment of the two sequences. The percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology can be evaluated using any of the variety of available sequence comparison algorithms and programs. Such algorithms and programs include, but are not limited to, TBLASTN, BLASTP, FASTA, TFASTA, CLUSTALW, FASTDB. In some cases, protein and nucleic acid sequence homologies can be evaluated using the Basic Local Alignment Search Tool (“BLAST”).

In some cases, the BBI protein may be modified, for example by glycosylation, lipidation, acetylation, phosphorylation, or oxidation. Modifications such as oxidation may enhance the activity of the BBI protein, such as chymotrypsin and/or trypsin inhibition. For example, oxidation of methionine at amino acid position 27 to a methionine sulfoxide may increase the trypsin and/or chymotrypsin inhibitory activity of the protein. In some cases, BBI proteins administered to treat disease comprise a methionine sulfoxide at amino acid position 27. Alternatively, a BBI protein may have one or more amino acid substitutions. The one or more amino acid substitutions may enhance certain biological properties of the protein, for example, the trypsin or chymotrypsin inhibitory activity. A soybean BBI protein administered to a patient may have, e.g., a Met27Val, Met27Leu, or Met27Ile substitution.

### IV. Peptide Administration

Peptide therapeutics described herein, including peptide hormones, peptide toxins, and enzyme inhibitors, may be administered as a single agent or as a combination. For example, a peptide hormone such as insulin, glucagon, glucagon-like peptide 1 (GLP-1), parathyroid hormone (PTH), relaxin-1, a relaxin-1 analogue such as single-chain relaxin-1, relaxin-2, a relaxin-2 analogue such as single-chain relaxin-2, relaxin-3, a relaxin-3 analogue such as single-chain relaxin-3, insulin-like peptide 3, insulin-like peptide 4, insulin-like peptide 5, insulin-like peptide 6, or fragments/variants thereof; a peptide toxin such as Mambalgin-1, Hg1, Stichodactyla toxin (ShK), or fragments/variants thereof; or an enzyme inhibitor such as soybean trypsin inhibitor (STI) protein, soybean Bowman-Birk inhibitor (BBI) protein, eglin C protein, or fragments/variant thereof may be administered as a single agent, such as with a pharmaceutically acceptable excipient, to treat a disease or disorder, for example an autoimmune disease or inflammatory condition.

The term “autoimmune disease,” as used herein, refers to the presence of an autoimmune response (an immune response directed against an auto- or self-antigen) in a subject. Autoimmune diseases include diseases caused by a breakdown of self-tolerance such that the adaptive immune system responds to self antigens and mediates cell and tissue damage. Autoimmune diseases may be characterized as being a result of, at least in part, a humoral immune response. Examples of autoimmune disease include, without limitation, acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplantation rejection, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)).

The term “inflammatory disease,” as used herein, refers to a disease resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. An inflammatory disease may comprise an antibody-mediated inflammatory process. An “inflammatory disease” can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's Syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, Crohn's Disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, and necrotizing enterocolitis. In a particular embodiment, the inflammatory disease is selected from the group consisting of atherosclerosis, arteriosclerosis, autoimmune disorders, multiple sclerosis, systemic lupus erythematosus, rheumatoid arthritis, inflammatory arthritis, and myocarditis.

Peptide therapeutics can be administered to treat various diseases, such as autoimmune and inflammatory diseases, via various routes including, but not limited to, parenteral routes such as intravenous injection, intra-arterial injection, intraosseous infusion, intra-muscular injection, intracerebral injection, intrathecal injection, and subcutaneous injection; enteral routes such as oral administration and rectal administration topical administration; and topical routes such as epicutaneous administration and nasal administration. Non-invasive methods of administration, such as oral and nasal administration, may be preferable to invasive methods of administration, such as injection, for considerations including patient comfort and compliance.

Oral delivery of peptide therapeutics may be considered minimally invasive and relatively easy to administer. However, the oral bioavailability of peptide therapeutics delivered orally may be lower than parenteral administration as peptide therapeutics may be subject to proteolysis in the gastrointestinal tract. The term “oral bioavailability” refers to the fraction of an orally administered drug that reaches systemic circulation. With intravenous administration, a drug, such as a peptide therapeutic, can be directly and fully available in the bloodstream and can be distributed via systemic circulation to the point where a pharmacological effect may occur place. A drug administered orally may need to cross further barriers to reach the systemic circulation, which can significantly reduce the final dosage of a drug in the bloodstream. A high oral bioavailability can reduce the amount of an administered drug necessary to achieve a desired pharmacological effect and therefore could reduce the risk of side-effects and toxicity. A poor oral bioavailability can result in low efficacy and higher inter-individual variability and therefore can lead to unpredictable response to a drug.

Various strategies may be employed to increase the bioavailability of orally administered drugs, such as chemical modification, formulation vehicles and use of enzyme inhibitors, absorption enhancers and mucoadhesive polymers. Enzyme inhibitors can be co-administered with peptide therapeutics to increase bioavailability by inhibiting the activity of proteases (e.g., trypsin, chymotrypsin, elastase, pepsin, and carboxypeptidases) which cleave amino acid side chains with varying specificity. Enzyme inhibitors may be more effective in the large intestine than the small intestine due to a larger quantity and variety of proteases within the small intestine. Examples of enzyme inhibitors include trypsin inhibitors, which are a type of serine protease inhibitor that reduces the biological activity of trypsin. Examples of trypsin inhibitors include soybean trypsin inhibitor, which is an inhibitor of chymotrypsin; Bowman-Birk inhibitor (BBI) proteins from legumes (e.g., soybean, pea, lentil, and chickpea); bovine pancreas trypsin inhibitor (BPTI); and ovomucin (trypsin inhibitor found in egg white, e.g., chicken egg white, duck egg white, and turkey egg white).

In some cases, peptide therapeutics described herein may be administered in combination. For example, a pharmaceutical composition comprising a soybean BBI protein may include a peptide. The peptide may be, for example, an insulin peptide, analogue or fragment thereof; a glucagon peptide, analogue or fragment thereof; and a glucagon-like peptide-1 (GLP-1) peptide, analogue or fragment thereof. In some cases, the BBI protein may have a methionine sulfoxide at amino acid position 27 that enhances its inhibitory activity. In some cases, the BBI protein may have an amino acid substitution, such as a Met27Val, Met27Leu, or Met27Ile substitution. The BBI protein may have at least 60% sequence identity (e.g., at least 65%, 70%, 75%, 80%, 85%, 90%, 95% sequence identity or greater) to SEQ ID NO: 1. The BBI protein administered may be a fragment of a BBI protein. Such pharmaceutical compositions (e.g., single peptide or combinations) may be formulated for oral delivery.

### V. Vectors

Also provided herein are vectors for producing peptide therapeutics. A vector may encode an expression tag, a cleavage tag, and/or a target peptide sequence. The expression tag may comprise an amino acid sequence having at least 80% sequence identity (e.g., 85%, 90%, 95%, or greater) to SEQ ID NO: 2 (MKAIFVLKGSLDRDPEFPSDKPHHKKHHKKHHSSGSLE). The expression tag may further comprise an affinity tag, such as charged tag for ion exchange affinity chromatography or capture. The affinity tag sequence may comprise a poly-histidine, a poly-lysine, poly-aspartic acid, poly-glutamic acid, or combinations thereof. A cleavage tag may facilitate selective chemical cleavage to yield a peptide of interest following purification. Such chemically cleavable amino acid sequences include Trp, His-Met, and Pro-Met. The vector may further comprise a nucleotide sequence that encodes an inclusion-directing peptide. Variously, the inclusion body targeting amino acid sequence comprises a peptide sequence derived from a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, and an inclusion-body directing functional homolog of BRCA2. In some embodiments, the vector further comprises an expression promoter located on the 5′ end of the affinity tag sequence.

A method for producing a peptide of commercial or therapeutic interest using a vector may comprise the steps of: a) cleaving a vector with a restriction endonuclease to produce a cleaved vector; b) ligating the cleavage site to one or more nucleic acids, wherein the nucleic acids encode a desired peptide having at least a base overhang at each end configured and arranged for ligation with the cleaved vector to produce a second vector suitable for expression of a fusion peptide; c) transforming the second vector into suitable host cell; d) incubating the host cell under conditions suitable for expression of the fusion peptide; e) isolation of inclusion bodies from the host cell; f) solubilization and extraction of the fusion peptide from the inclusion bodies; g) binding of the fusion peptide to a suitable affinity material; h) optionally, washing of bound fusion peptide to remove impurities; and i) cleaving the fusion peptide to release the said target peptide.

Methods of producing peptides described herein may provide a high yield of peptide with high purity, such as a purity of at least 95% (e.g., at least 96%, 97%, 98%, 99% purity or greater). Peptides produced may be R&D grade peptides or clinical grade therapeutics. Such peptides may include insulin, glucagon, glucagon-like peptide 1 (GLP-1), parathyroid hormone 1-34 (PTH-34), relaxin-1, relaxin-1 analogues such as single-chain relaxin-1, relaxin-2, relaxin-2 analogues such as single-chain relaxin-2, relaxin-3, relaxin-3 analogues such as single-chain relaxin-3, insulin-like peptide 3, insulin-like peptide 4, insulin-like peptide 5, insulin-like peptide 6, soybean trypsin inhibitor (STI) protein, soybean Bowman-Birk inhibitor (BBI) protein, eglin C protein, Mambalgin-1, Hg1 toxin, and Stichodactyla toxin (ShK).

### VI. Target Peptides

The methods for producing peptides described herein are applicable to a wide range of peptides as the isolated product, which may be referred to as target peptides. Peptides produced may be naturally-occurring peptides, non-naturally-occurring peptides, or naturally-occurring peptides with non-natural substitutions, deletions, or additions. The target peptide may be modified chemically or biologically following isolation to yield a derivative of the target peptide.

The peptide may be a vaccine, an antibody, a recombinant hormone and protein, interferon, interleukin, or growth factor. The target peptide may be fifty or fewer amino acids in length. In some cases, the target peptide may be greater than fifty amino acids in length.

Non-limiting examples of peptides that can be produced using methods described herein include peptides, analogs, and fragments thereof selected from the group consisting of angiotensin, arginine vasopressin (AVP), AGG01, amylin (IAPP), amyloid beta, N-acetylgalactosamine-4-sulfatase (rhASB; galsulfase), avian pancreatic polypeptide (APP), B-type natriuretic peptide (BNP), calcitonin peptides, calcitonin, colistin (polymyxin E), colistin copolymer 1 (Cop-1), cyclosporin, darbepoetin, PDpoetin, dornase alfa, eledoisin, β-endorphin, enfuvirtide, enkephalin pentapeptides, epoetin, epoetin delta, erythropoietin, exenatide, factor VIII, factor X, follicle-stimulating hormone (FSH), alpha-galactosidase A (Fabrazyme), Growth Hormone Releasing Hormone 1-24 (GHRH 1-24), (3-globin, glucagon, glucocerebrosidase, glucagon-like peptide-1 (GLP-1), granulocyte-colony stimulating factor (G-CSF), granulocyte macrophage-colony stimulating factor (GM-CSF), growth hormone, Hepatitis B viral envelope protein, human growth hormone (hGH), insulin, insulin A, insulin B, insulin-like growth factor 1 (IGF-1), interferon, kassinin, alpha-L-iduronidase (rhIDU; laronidase), lactotripeptides, leptin, liraglutide (NN2211, VICTOZA), luteinizing-hormone-releasing hormone, methoxy polyethylene glycol-epoetin beta (MIRCERA), myoglobin, neurokinin A, neurokinin B, NN9924, NPY (NeuroPeptide Y), octreotide, pituitary adenylate cyclase activating peptide (PACAP), parathyroid hormone (PTH), Peptide Histidine Isoleucine 27 (PHI 27), proopiomelanocortin (POMC) peptides, prodynorphin peptides, polymyxins, polymyxin B, Pancreatic PolYpeptide (PPY), Peptide YY (PYY), secretin, somatostatin, Substance P, teriparatide (FORTEO), tissue plasminogen activator (TPA), thrombospondins (TSP), ubiquitin, urogastrone, Vasoactive Intestinal Peptide (VIP, or PHM27), and viral envelope proteins. In various embodiments, the target peptide is selected from amyloid beta, calcitonin, enfuvirtide, epoetin, epoetin delta, erythropoietin, exenatide, factor VIII, factor X, glucocerebrosidase, glucagon-like peptide-1 (GLP-1), granulocyte-colony stimulating factor (G-CSF), human growth hormone (hGH), insulin, insulin A, insulin B, insulin-like growth factor 1 (IGF-1), interferon, liraglutide, somatostatin, teriparatide, and tissue plasminogen activator (TPA). In various embodiments, the target peptide is selected from amyloid beta and insulin.

The target peptide may be a hormone. For example, the target peptide may be selected from the group consisting of Activin, inhibin, Adiponectin, Adipose derived hormones, Adrenocorticotropic hormone, Afamelanotide, Agouti signalling peptide, Allatostatin, Amylin, Angiotensin, Atrial natriuretic peptide, Bovine somatotropin, Bradykinin, Brain-derived neurotrophic factor, CJC-1295, Calcitonin, Ciliary neurotrophic factor, Corticotropin-releasing hormone, Cosyntropin, Endothelin, Enteroglucagon, Follicle-stimulating hormone, Gastrin, Gastroinhibitory peptide, Glucagon, Glucagon hormone family, Glucagon-like peptide-1, Gonadotropin, Granulocyte colony-stimulating factor, Growth hormone, Growth hormone releasing hormone, Hepcidin, Human chorionic gonadotropin, Human placental lactogen, Incretin, Insulin, Insulin glargine, Insulin lispro, Insulin aspart, Insulin-like growth factor 2, Insulin-like growth factor, Leptin, Liraglutide, Luteinizing hormone, Melanocortin, Melanocyte-stimulating hormone, Melanotan II, Minigastrin, N-terminal prohormone of brain natriuretic peptide, Nerve growth factor, Neurotrophin-3, NPH insulin, Obestatin, Orexin, Osteocalcin, Pancreatic hormone, Parathyroid hormone, Peptide YY, Peptide hormone, Plasma renin activity, Pramlintide, Preprohormone, Proislet Amyloid Polypeptide, Prolactin, Relaxin, Renin, Salcatonin, Secretin, Sincalide, Teleost leptins, Thyroid-stimulating hormone, Thyrotropin-releasing hormone, Urocortin, Urocortin II, Urocortin III, Vasoactive intestinal peptide, and Vitellogenin.

In some cases, the target peptide does not include tryptophan in their sequence.

### VII. Inclusion-Body Directing Peptides

Inclusion bodies are composed of insoluble and denatured forms of a peptide and are about 0.5-1.3 μm in diameter. These dense and porous aggregates may help to simplify recombinant protein production since they may have a high homogeneity of the expressed protein or peptide, can result in lower degradation of the expressed protein or peptide because of a higher resistance to proteolytic attack by cellular proteases, and may be easy to isolate from the rest of the cell due to differences in their density and size relative to the other cellular components. In various embodiments, the presence of inclusion bodies permits production of increased concentrations of the expressed protein or peptide due to reduced toxicity by the protein or peptide upon segregation into an inclusion body. Once isolated, the inclusion bodies may be solubilized to allow for further manipulation and/or purification. An inclusion-body directing peptide is an amino acid sequence that helps to direct a newly translated protein or peptide into insoluble aggregates within the host cell. Prior to final isolation, a target peptide may be produced as a fusion peptide where the fusion peptide includes as part of its sequence of amino acids an inclusion-body directing peptide. Non-limiting examples of inclusion-body directing peptides include a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, and an inclusion-body directing functional homolog of BRCA2.

### VIII. Affinity Tag Peptides

A wide variety of affinity tags may be used. Affinity tags may be specific for cations, anions, metals, or any other material suitable for an affinity column. In some cases, any peptide not possessing an affinity tag will elute through the affinity column leaving the desired fusion peptide bound to the affinity column via the affinity tag.

Specific affinity tags may include poly-lysine, poly-histidine, poly-glutamic acid, poly-arginine peptides, or combinations thereof. For example, the affinity tags may be 5-10 lysines (SEQ ID NO: 5), 5-10 histidines (SEQ ID NO: 6), 5-10 glutamic acids (SEQ ID NO: 7), or 5-10 arginines (SEQ ID NO: 8). In some cases, the affinity tag is a hexa-histidine sequence (SEQ ID NO: 4), hexa-lysine sequence (SEQ ID NO: 9), hexa-glutamic acid sequence (SEQ ID NO: 10), or hexa-arginine sequence (SEQ ID NO: 11). Alternatively, the HAT-tag (Clontech) may be used. In some cases, the affinity tag is a His-Trp Ni-affinity tag.

Without wishing to be bound by theory, it is believed that the histidine residues of a poly-histidine tag bind with high affinity to Ni-NTA or TALON resins. Both of these resins contain a divalent cation (Ni-NTA resins contain Mg²⁺; TALON resins contain Co²⁺) that forms a high affinity coordination with the His tag.

The affinity tag may have a pI (isoelectric point) that is at least one pH unit separate from the pI of the target peptide. Such difference may be either above or below the pI of the target peptide. For example, the target peptide has a high pI and the affinity tag has a pI that is at least one pH unit lower, at least two pH units lower, at least three pH units lower, at least four pH units lower, at least five pH units lower, at least six pH units lower, or at least seven pH units lower. Alternatively, the target peptide has a low pI, and the affinity tag has a pI that is at least one pH unit higher, at least two pH units higher, at least three pH units higher, at least four pH units higher, at least five pH units higher, at least six pH units higher, or at least seven pH units higher. In some cases, the target peptide has a pI of about 10 and the affinity tag has a pI of about 6.

The affinity tag may be contained within the native sequence of the inclusion body directing peptide. Alternatively, the inclusion body directing peptide can be modified to include an affinity tag. For example, the affinity tag can be a KSI or BRCA2 sequence modified to include extra histidines, extra lysines, extra arginines, extra glutamic acids, or combinations thereof.

In some cases, affinity tags are epitopes such as FLAG (Eastman Kodak) or myc (Invitrogen) that can be used in conjunction with their antibody pairs.

### IX. Cleavage Tags

A wide range of cleavage tags may be used. In some cases, the cleavage tag is a tryptophan at the amino terminus of the target peptide. Upon cleavage with a cleaving agent, the amide bond connecting the tryptophan to the target peptide is cleaved, and the target peptide is released from the affinity column.

Alternatively, the cleavage tag may be a tryptophan at the amino terminus of the target peptide, where the cleavage tag also includes an amino acid with a charged side-chain in the local environment of the tryptophan, such as within five amino acids on the upstream (i.e. amino) or downstream (i.e. carboxy) side of the tryptophan. The presence of an amino acid side-chain within five amino acids on the amino terminus of the tryptophan amino acid allows for selectivity of cleavage of the tryptophan of the cleavable tag over any other tryptophans that may be present in the heterologous fusion peptide, for example, tryptophans as part of the inclusion body directing peptide or as part of the target peptide. In some cases, an amino acid with a positively charged side chain such as lysine, ornithine, or arginine is within five, four, three, or two amino acid units, or is adjacent on the amino terminus to the tryptophan of the cleavable tag. In some cases, a glutamic acid amino acid is within five, four, three, or two amino acid units, or is adjacent on the amino terminus to the tryptophan of the cleavable tag.

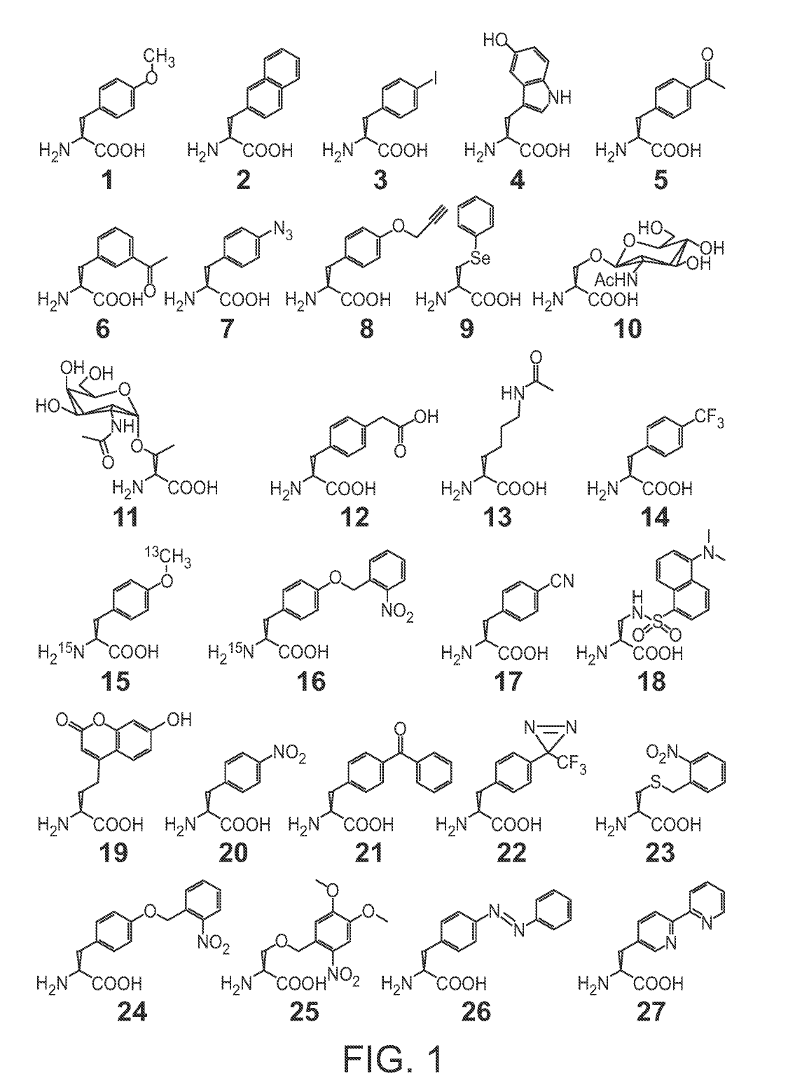

The cleavage tag may be His-Met or Pro-Met. In some cases, the cleavage tag is an unnatural amino acid. Cells have been modified to enable the cells to produce peptides which contain unnatural amino acids. For instance, Wang, et al., (2001) Science 292:498-500, describes modifications made to the protein biosynthetic machinery of E. coli which allow the site-specific incorporation of an unnatural amino acid, O-methyl-L-tyrosine, in response to an amber stop codon (TAG). Wang, et al., (2009) Chem Biol. 16(3):323-36 provides a review of numerous unnatural amino acids that have been site-specifically incorporated into proteins in E. coli, yeast, or mammalian cells. Without wishing to be bound by theory, it is believed that incorporation of one or more unnatural amino acids can provide additional selectivity for cleavage at the unnatural amino acid over non-specific cleavage at other sites on the fusion peptide. In various embodiments, the unnatural amino acid is selected from compounds 1-27 in FIG. 1.

Heterologous fusion peptides produced by methods described herein may comprise unnatural amino acids. In some aspects, prokaryotic cells with modifications to the protein biosynthetic machinery produce such fusion peptides. Examples of such prokaryotic cells include E. coli. In some aspects the modifications comprise adding orthogonal tRNA/synthetase pairs. In some aspects four base codons encode novel amino acids. In some aspects, E. coli allow the site-specific incorporation of the unnatural amino acid O-methyl-L-tyrosine into a peptide in response to an amber stop codon (TAG) being included in an expression vector.

### X. Fusion Peptide Synthesis

Various methods may be employed to produce peptides, including peptide therapeutics. Various non-limiting methods are described in detail herein.

### A. Ribosomal Synthesis

Peptides may be produced by ribosomal synthesis, which utilizes transcription and translation to express peptides. Some peptides can be expressed in their native form in eukaryotic hosts, such as mammalian cell systems (e.g., Chinese hamster ovary (CHO) cells, human embryonic kidney (HEK) cells including HEK 293 and HEK 293F cells, HeLa cells, PC3 cells, Vero cells, and MC3T3 cells); yeast cell systems (e.g., Saccharomyces cerevisiae, Bacillus subtillis, and Pichia pastoris); and insect cell systems (e.g., Sf9, Sf21, and High Five strains). As an alternative, bacterial host expression systems, such as systems using Escherichia coli (E. coli), Corynebacterium, and Pseudomonas fluorescens, can be used.

A nucleic acid sequence, such as a DNA sequence, which can serve as a template for transcription in ribosomal synthesis may be provided in a vector. A vector may provide additional nucleotide sequences useful for protein expression via ribosomal synthesis. A vector generally refers to one or more nucleotide sequences that are operably linked. The term “operably linked,” as used herein, refers to nucleotide sequences placed in a functional relationship with another nucleotide sequence. Nucleotide sequences of a vector can encode for a protein (e.g., protein coding sequence) such as a target peptide or may comprise vector elements such as control or regulatory sequences, selectable markers, promoters (e.g. inducible and constitutive), ribosomal binding sites, termination sequences, etc. Selectable markers, such as antibiotic resistance, may enable selective screening against the cells that do not contain the constructed vector with the gene of interest. Vectors may include hybrid promoters and multiple cloning sites for the incorporation of different genes. A vector may also include a nucleotide sequence encoding an expression tag and/or a cleavage tag. Non-limiting examples of expression vectors include the pET system and the pBAD system (e.g., for bacterial expression systems); the pPIC system and the pYES system (e.g., for yeast expression systems); and the pcDNA system (e.g., for mammalian expression systems). The choice of nucleic acid vector and vector elements can be chosen for compatibility with the host expression system.

For example, the pET system can encompass more than 40 different variations on the standard pET vector. In some cases, the pET system may utilize a T7 promoter that is recognized specifically by T7 RNA polymerase. This polymerase can transcribe DNA five times faster than E. coli RNA polymerase, allowing for increased levels of transcription.

A vector may be designed to include sequences encoding for a heterologous fusion peptide comprising an expression tag such as SEQ ID NO: 2 (MKAIFVLKGSLDRDPEFPSDKPHHKKHHKKHHSSGSLE), a cleavage tag, and a target peptide. In some cases, the vector further comprises nucleotide sequences encoding for an inclusion body directing peptide and/or an affinity tag. An affinity tag, such as, but not limited to, a sequence of charged amino acids (e.g. polyhistidine and/or polylysine), an AviTag, a FLAG-tag, an HA-tag, a Myc-tag, an SBP-tag, or combinations thereof, may also be included in the expression tag and used for purification processes. For example, a pET-19b vector to be used with bacterial expression systems may comprise nucleotide sequences encoding for an expression tag such as SEQ ID NO: 2 (MKAIFVLKGSLDRDPEFPSDKPHHKKHHKKHHSSGSLE) or a fragment thereof, a cleavage tag, a target peptide such as a BBI protein, and optionally an inclusion body directing peptide and/or an affinity tag Similarly, a pPIC vector to be used with yeast expression systems or a pcDNA vector to be used with mammalian expression systems may comprise nucleotide sequences encoding for an expression tag such as SEQ ID NO: 2 (MKAIFVLKGSLDRDPEFPSDKPHHKKHHKKHHSSGSLE) or a fragment thereof, a cleavage, a target peptide such as a BBI protein, and optionally a inclusion body directing peptide and/or an affinity tag.

The vector may be introduced into a host cell, such as a bacterial cell (e.g., E. coli, Corynebacterium, and Pseudomonas fluorescens or a yeast cell (e.g., Saccharomyces cerevisiae, Bacillus subtillis, and Pichia pastoris), using any suitable method, including transformation, transfection, electroporation, and microinjection. For example, transformed E. coli cells can be plated onto agar containing an antibacterial agent to prevent the growth of any cells that do not contain a resistance gene, thereby selecting for cells that have been transformed. Colonies from the plating process may be grown in starter culture or broth according to standard cell culture techniques. For example, one colony from an agar plate is grown in a starter culture of broth, which may optionally contain an antibacterial agent. Typically, cells are grown to a preselected optical density before being further processed to obtain fusion peptide. For example, cells may be grown to an optical density (OD) of 0.1, 0.2, 0.3, 0.4, 0.5, 0.6, 0.7, 0.8, or 0.9, all values being about. In some embodiments the cells are grown to an optical density (OD) of about 0.5.

Once a vector is introduced into a host cell, the host cell may be used for heterologous peptide expression. With a vector comprising a constitutive promoter, expression of the heterologous fusion peptide may occur when the vector is introduced into the host cell. With a vector comprising an inducible promoter, expression of the desired heterologous fusion peptide may be induced or activated in a cell having a vector, for example using molecules that can activate an inducible promoter. For example, in E. coli cells, the lac operon can serve as an inducible promoter that is activated under certain environmental conditions. E. coli are always capable of metabolizing the monosaccharide glucose. However, in order to metabolize the disaccharide lactose, the cells need an enzyme known as β-galactosidase. Thus, low extracellular glucose concentrations and high lactose concentrations induce the lac operon and the gene for β-galactosidase is transcribed. In some cases, an inducible promoter such as the lac operon is situated upstream from the sequence coding for the fusion peptide. Upon induction of the lac operon, transcription of the sequence coding for the desired fusion peptide occurs.

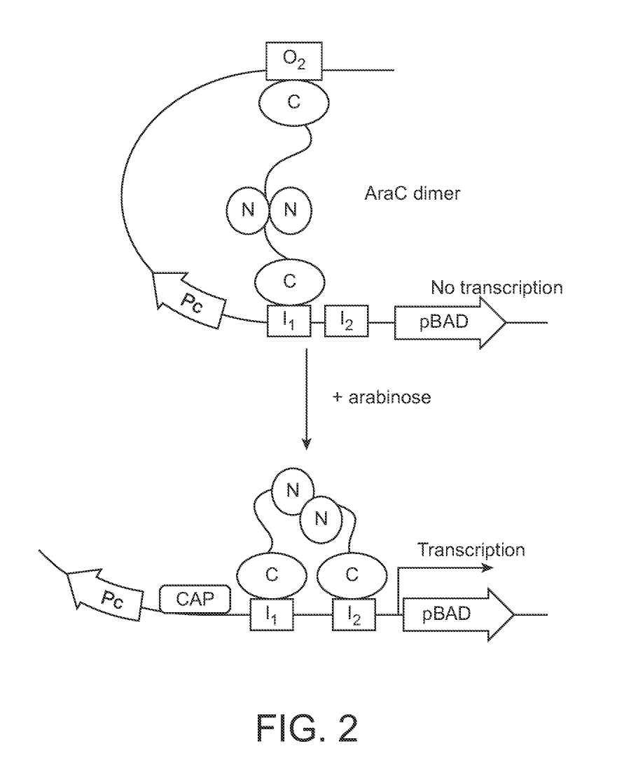

The term “activation” refers to the removal of repressor protein. A repressor protein is generally allosteric meaning it changes shape when bound by an inducer molecule and dissociates from the promoter. This dissociation allows for the transcription complex to assemble on DNA and initiate transcription of any genes downstream of the promoter. Therefore, by splicing genes produced in vitro into the bacterial genome, one can control the expression of novel genes. This trait may be used advantageously when dealing with inclusion bodies if the production and amassing of inclusion bodies becomes toxic enough to kill E. coli. For example, expression of the desired fusion peptide can be delayed until a sufficient population of cells has been cultured, and then the promoter can be induced to express a large amount of fusion peptide by removal of the repressor protein. Thus, the L-arabinose operon may be activated according to the invention for increased protein expression at a desired timepoint. Specifically, the L-arabinose operon may be activated by both the addition of L-arabinose into the growth medium and the addition of IPTG, a molecule that acts as an activator to dissociate the repressor protein from the operator DNA. FIG. 2 illustrates one embodiment of the activation of transcription in a pBAD vector via the addition of L-arabinose. Without wishing to be bound by theory, it is believed that L-arabinose binds to the AraC dimer causing the protein to release the O₂ site on the DNA and bind to the I₂ site. These steps serve to release the DNA loop and enable its transcription. Additionally, the cAMP activator protein (CAP) complex stimulates AraC binding to I₁ and I₂—a process initiated with IPTG.

In some cases, cells expressing only a fusion peptide with an expression tag, a cleavage tag, and the target peptide may not be able to produce large amounts of fusion peptide. The reasons for low production yields may vary. For example, the heterologous fusion peptide may be toxic to the host cell (e.g., the bacterial cell), thus causing the host cell to die upon production of certain levels of the fusion peptide. Alternatively, the target peptide may be either poorly expressed or rapidly degraded in the bacterial system. In some cases, the target peptide may be modified by the host cell, including modifications such as glycosylation. To remedy some or all of these problems, the desired fusion peptide may be directed to an inclusion body, thereby physically segregating the target peptide from degradative factors in the cell's cytoplasm or, in the case of target peptides that are toxic to the host such as peptide antibiotics, physically segregating the target peptide to avoid toxic effects on the host. Moreover, by physically aggregating the fusion peptide in an inclusion body, the subsequent separation of the fusion peptide from the constituents of the host cell and the media (i.e., cell culture or broth) may be performed more easily or efficiently. In some cases, the host cell may be modified for increased protein expression efficiency. For example, a bacterial cell, such as an E. coli, cell may be modified to be protease deficient.

Target peptides may be directed to inclusion bodies by an inclusion-body directing peptide as part of the heterologous fusion peptide. In some cases, an otherwise identical heterologous fusion peptide without an inclusion-body directing peptide has minimal or no tendency to be directed to inclusion bodies in an expression system. Alternatively, an otherwise identical heterologous fusion peptide without an inclusion-body directing peptide has some tendency to be directed to inclusion bodies in an expression system, but the number, volume, or weight of inclusion bodies is increased by producing a fusion peptide with an inclusion-body directing peptide. In some cases, where the target peptide itself directs the fusion peptide of the invention to inclusion bodies, a separate inclusion-body directing peptide may be excluded.

For example, methods have been described which allow α-human atrial natriuretic peptide (a-hANP) to be synthesized in stable form in E. coli. Eight copies of the synthetic α-hANP gene were linked in tandem, separated by codons specifying a four amino acid linker with lysine residues flanking the authentic N and C-termini of the 28 amino acid hormone. That sequence was then joined to the 3′ end of the fragment containing the lac promoter and the leader sequence coding for the first seven N terminal amino acids of β-galactosidase. The expressed multidomain protein accumulated intracellularly into stable inclusion bodies and was purified by urea extraction of the insoluble cell fraction. The purified protein was cleaved into monomers by digestion with endoproteinase lys C and trimmed to expose the authentic C-terminus by digestion with carboxypeptidase B. See Lennick et al., “High-level expression of α-human atrial natriuretic peptide from multiple joined genes in Escherichia coli,” Gene, 61:103-112 (1987), incorporated by reference herein.

Directing the target peptide to an inclusion body by producing the target peptide as part of a fusion peptide may lead to higher output of peptide. For example, the desired fusion peptide may be produced in concentrations greater than 100 mg/L. The desired fusion peptide may be produced in concentrations greater than about 200 mg/L, 250 mg/L, 300 mg/L, 350 mg/L, 400 mg/L, 450 mg/L, 500 mg/L, 550 mg/L, 600 mg/L, 650 mg/L, 700 mg/L, 750 mg/L, 800 mg/L, 850 mg/L, 900 mg/L, 950 mg/L, and 1 g/L, all amounts being prefaced by “greater than about.” In some cases, the output of desired fusion peptide is greater than about 1.5 g/L, greater than about 2 g/L, or greater than about 2.5 g/L. The output of desired fusion peptide may be in the range of from about 500 mg/L to about 2 g/L, or from about 1 g/L to about 2.5 g/L. In some cases, the desired fusion peptide is produced in yields equal to or greater than 500 mg/L of media.

The inclusion-body directing peptide may be a ketosteroid isomerase (KSI) or inclusion-body directing functional fragment thereof. The inclusion-body directing functional fragment may have at least 25, at least 30, at least 35, at least 40, at least 45, at least 50, at least 55, at least 60, at least 65, at least 70, at least 75, at least 80, at least 85, at least 90, at least 95, or at least 100 amino acids. Homologs of a ketosteroid isomerase are also encompassed. Such homologs may have at least 25, at least 30, at least 35, at least 40, at least 45, at least 50, at least 55, at least 60, at least 65, at least 70, at least 75, at least 80, at least 85, at least 90, or at least 95 percent sequence identity with the amino acid sequence of a ketosteroid isomerase. An expression system for a fusion peptide with a functional fragment or homolog of a ketosteroid isomerase may produce at least 25, at least 30, at least 35, at least 40, at least 45, at least 50, at least 55, at least 60, at least 65, at least 70, at least 75, at least 80, at least 85, at least 90, at least 95, or greater than 100 percent of the amount of inclusion bodies produced by an otherwise identical expression system with a fusion peptide containing a complete ketosteroid isomerase peptide sequence.

### B. Synthetic Peptide Synthesis

The heterologous fusion peptide can alternatively be made through solid phase peptide synthesis (SPPS) or liquid-phase peptide synthesis. SPPS involves covalently linking amino acids in an ordered manner to form a synthetic peptide with a desired amino acid sequence. Solid supports, e.g., polystyrene resin, polyamide resin, polyethylene (PEG) hybrid polystyrene resin, or PEG-based resin, are provided as a structural support for the elongation of the peptide, generally from the C-terminus to the N-terminus. Amino acids with “temporary” protecting groups, e.g., 9-fluorenylmethyloxycarbonyl group (Fmoc) or t-butyloxycarbonyl (Boc) protecting groups, are added to the N-terminus of a growing peptide chain through iterations of various steps including deprotection, e.g., removal of protecting groups, and reaction steps, e.g., formation of peptide bonds.

Liquid-phase peptide synthesis similarly adds amino acids to a growing peptide chain in an ordered fashion, however, without the aid of a solid support. Liquid-phase peptide synthesis generally requires that the C-terminus of the first amino acid be protected and the growing peptide chain be isolated from the reaction reagents after each amino acid addition so that one amino acid is not unintentionally incorporated two or more times into the peptide chain.

In one embodiment, the solid phase peptide synthesis uses Fmoc protecting groups. The Fmoc protecting group utilizes a base labile alpha-amino protecting group. In an alternative embodiment, the solid phase peptide synthesis uses Boc protecting groups. The Boc protecting group is an acid labile alpha-amino protecting group. Each method may involve distinct resin addition, amino acid side-chain protection, and consequent cleavage/deprotection steps. Generally, Fmoc chemistry generates peptides of higher quality and in greater yield than Boc chemistry. Impurities in Boc-synthesized peptides are mostly attributed to cleavage problems, dehydration and t-butylation. Once assembled on the solid support, the peptide is cleaved from the resin using strongly acidic conditions, usually with the application of trifluoracetic acid (TFA). It is then purified using reverse phase high pressure liquid chromatography, or RP-HPLC, a process in which sample is extruded through a densely packed column and the amount of time it takes for different samples to pass through the column (known as a retention time) is recorded. As such, impurities are separated out from the sample based on the principle that smaller peptides pass through the column with shorter retention times and vice versa. Thus, the protein being purified elutes with a characteristic retention time that differs from the rest of the impurities in the sample, thus providing separation of the desired protein. Other examples of purification techniques include size exclusion chromatography (SEC) and ion exchange chromatography (IEC).

Solid-phase peptide synthesis generally provides high yields because excess reagents can be used to force reactions to completion. Separation of soluble byproducts is simplified by the attachment of the peptide to the insoluble support throughout the synthesis. Because the synthesis occurs in the same vessel for the entire process, mechanical loss of material is low.

In various embodiments, an inclusion body directing peptide may be excluded. Alternatively, an inclusion body directing peptide may be included to provide beneficial folding properties and/or solubility/aggregating properties.

### C. Non-Ribosomal Synthesis

Peptides may be produced by non-ribosomal synthesis. Such peptides include circular peptides and/or depsipeptides. Nonribosomal peptides can be synthesized by one or more nonribosomal peptide synthetase (NRPS) enzymes. These enzymes are independent of messenger RNA. Nonribosomal peptides often have a cyclic and/or branched structure, can contain non-proteinogenic amino acids including D-amino acids, carry modifications like N-methyl and N-formyl groups, or are glycosylated, acylated, halogenated, or hydroxylated. Cyclization of amino acids against the peptide backbone is often performed, resulting in oxazolines and thiazolines; these can be further oxidized or reduced. On occasion, dehydration is performed on serines, resulting in dehydroalanine.

The enzymes of an NRPS are organized in modules that are responsible for the introduction of one additional amino acid. Each module consists of several domains with defined functions, separated by short spacer regions of about 15 amino acids. While not wishing to be bound by theory, it is thought that a typical NRPS module is organized as follows: initiation module, one or more elongation modules, and a termination module. The NRPS genes for a certain peptide are usually organized in one operon in bacteria and in gene clusters in eukaryotes.

In some cases, an inclusion body directing peptide may be excluded. Alternatively, an inclusion body directing peptide may be included to provide beneficial folding properties and/or solubility/aggregating properties.

XI. Separation of Fusion Peptide from Formation Media

Following production of the desired heterologous fusion peptides (e.g., in host cell expression systems), separation from the production media may be required. Optionally, following separation, the desired fusion peptide and carrier may be concentrated to remove excess liquid. Numerous methods for separating fusion peptides from their formation media and subsequent handling may be adapted and used. Various methods are described in detail herein.

### A. Fusion Peptides Targeted to Inclusion Bodies

The cells used to produce the desired fusion peptides may be lysed to release the fusion peptides. For example, where the desired fusion peptide is aggregated in inclusion bodies, the cell may by lysed, followed by separation of the inclusion bodies from the production media and cellular detritus. Any appropriate method of cell lysis may be used, including chemical lysis and mechanical lysis.

For example, cells can be disrupted using high-power sonication in a lysis buffer. A lysis buffer containing Tris, sodium chloride, glycerol, and a protease inhibitor may be added before lysis. In some cases, a lysis buffer containing about 25 mM Tris pH 8.0, about 50 mM NaCl, about 10% glycerol, and the protease inhibitor 1000X PMSF may added before lysis. Insoluble inclusion bodies may be collected using one or more washing steps and centrifugation steps. Wash buffers may include any reagents used for the stabilization and isolation of proteins. For example, wash buffers used may contain varying concentrations of Tris pH 8.0, NaCl, and Triton X100.

Targeting the desired fusion peptide to an inclusion body may result in higher initial purity upon lysis of the cell. For example, lysis of the cell and isolation of inclusion bodies through physical means such as centrifugation may result in an initial purity of greater than about 70%, great than about 75%, greater than about 80%, greater than about 85%, greater than about 90%, or greater than about 95% for the desired fusion peptide.

In some cases, following cell lysis, inclusion bodies form a pellet and remain in the pellet rather than supernatant until a solubilization step. The pellet can be washed clean of the remaining cellular components, and insoluble inclusion bodies are solubilized in a buffer for further handling. Solubilization buffers may include urea or any other chaotropic agent necessary to solubilize the fusion peptide. Without wishing to be bound by theory, it is believed that the solubilization step involves solubilizing the inclusion bodies in a chaotropic agent which serves to disrupt the peptides by interfering with any stabilizing intra-molecular interactions.

The solubilization buffer may include urea, guanidinium salts, or organic solvents. For example, a solubilization buffer may contain about 25 mM Tris pH 8.0, about 50 mM, NaCl, about 0.1 mM PMSF, and about 8M urea. In some cases, solubilization of inclusion bodies occurs with the addition of 8M urea as the sole chaotropic agent, and other chaotropic agents are excluded. Alternatively, the solubilization buffer may exclude urea or guanidinium salts. For example, guanidinium salts may be excluded to avoid interference with further processing on an ion exchange column. As an additional example, high urea concentrations such as about 8M urea may be excluded to avoid denaturing proteases that may be included in the solubilization buffer.

In some cases, a minimal amount of solubilization buffer is used. In the event that excess solubilization buffer is present, the solution may be processed to remove excess solvent prior to further purification.

### B. Fusion Peptides Not Targeted to Inclusion Bodies

In some cases, fusion peptides are not directed to inclusion bodies. The fusion peptides may remain in the cytosol of the cell, or the fusion peptides according to the invention may be secreted from the cell. Soluble fusion peptides may be isolated by any method, such as centrifugation, gel electrophoresis, pH or ion exchange chromatography, size exclusion chromatography, reversed-phase chromatography, dialysis, osmosis, filtration, and extraction.

### XII. Purification by Affinity Chromatography

Following cell lysis and initial isolation and solubilization of fusion peptides, the fusion peptides may be further purified by affinity chromatography, which is a highly selective process that relies on biologically-relevant interactions between an immobilized stationary phase and the fusion peptide to be purified. In some cases, the immobilized stationary phase is a resin or matrix. Without wishing to be bound by theory, it is believed that affinity chromatography functions by selective binding of the desired component from a mixture to the immobilized stationary phase, followed by washing of the stationary phase to remove any unbound material.

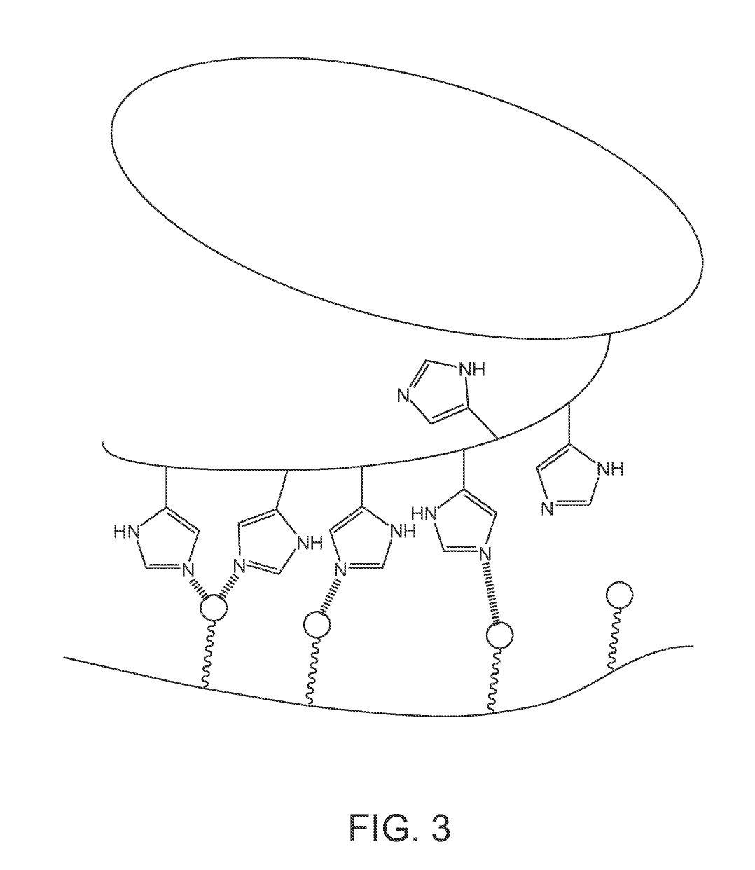

A wide variety of affinity chromatography systems may be used. For example, polyhistidine binds with great affinity and specificity to nickel and thus an affinity column of nickel, such as QIAGEN nickel columns, can be used for purification. Alternatively, Ni-NTA affinity chromatography resin (available from Invitrogen) may be used. FIG. 3 provides a schematic of an example of an immobilized Ni—NTA resin binding to a 6× HisTag (SEQ ID NO: 4) on a protein. Metal affinity chromatography has been used as a basis for protein separations, wherein a specific metal chelating peptide on the N- or C-terminus of a protein that can be used to purify that protein using immobilized metal ion affinity chromatography.

The affinity column can first be equilibrated with a buffer which may be the same as used for the solubilization of the fusion peptide. The column can then be charged with the solubilized fusion peptide, and buffer is collected as it flows through the column. In some cases, the column may be washed successively to remove urea and/or other impurities such as endotoxins, polysaccharides, and residual contaminants remaining from the cell expression system.

XIII. Removal of Target Peptide from Affinity Column via Cleavage

Numerous methods for cleavage of the fusion peptides on the affinity column may be used. In general, the cleavage step may occur by introduction of a cleavage agent which interacts with the cleavage tag of the fusion peptide resulting in cleavage of the heterologous fusion peptide and release of the target peptide. Following cleavage, the affinity column may be flushed to elute the target peptide while the portion of the fusion peptide containing the affinity tag remains bound to the affinity column. Following elution of the target peptide, the eluting solution may be concentrated to a desired concentration. The target peptide may be further processed and/or packaged for distribution or sale.

Control of the cleavage reaction may occur through chemical selectivity. For example, the cleavage tag may include a unique chemical moiety which is absent from the remainder of the fusion peptide such that the cleavage agent selectively interacts with the unique chemical moiety of the cleavage tag. In some cases, control of the cleavage reaction occurs through a unique local environment. For example, the cleavage tag may include a chemical moiety that is present elsewhere in the fusion peptide, but the local environment differs resulting in a selective cleavage reaction at the cleavage tag. For example, the cleavage tag includes a tryptophan and a charged amino acid side chain within five amino acids of the tryptophan. In some cases, the charged amino acid is on the amino terminus of the tryptophan amino acid.

Control of the cleavage reaction may alternatively occur through secondary or tertiary structure of the fusion peptide. For example, where identical moieties are present in the cleavage tag and elsewhere in the fusion peptide, the other portions of the fusion peptide may fold in secondary or tertiary structure such as alpha-helices, beta-sheets, and the like, to physically protect the susceptible moiety, resulting in selective cleavage at the cleavage tag.

Minor or even major differences in selectivity of the cleavage reaction for the cleavage tag over other locations in the fusion peptide may be amplified by controlling the kinetics of the cleavage reaction. For example, the concentration of cleavage agent can be controlled by adjusting the flow rate of eluting solvent containing cleavage agent. In some cases, the concentration of cleavage agent is maintained at a low level to amplify differences in selectivity. The reservoir for receiving the eluting solvent may contain a quenching agent to stop further cleavage of target peptide that has been released from the column.

Moreover, various methods for removal of peptides from affinity columns may be excluded. For example, washing an affinity column with a solution of a compound with competing affinity in the absence of a cleavage reaction may be excluded. In some cases, the step of washing an affinity column with a solution of imidazole as a displacing agent to assist in removing a fusion peptide from an affinity column is specifically excluded.

In some cases, multiple cleavages may occur. For example, insulin is naturally produced from a proinsulin precursor requiring two cleavage events. Both cleavage events may be required in order for the mature insulin to be properly folded. Therefore, a vector designed for insulin production may comprise two cleavage tags. Preferably, when more than one cleavage tag is present, the distinct cleavage tags are orthogonal, or able to be cleaved with specificity by different cleavage agents. For example, one cleavage tag may be a methionine amino acid while the other cleavage tag may be a tryptophan amino acid.

Non-limiting examples of cleavage agents include NBS, NCS, cyanogen bromide, Pd(H₂O)₄2-ortho iodobenzoic acid, DMSO/sulfuric acid, or a proteolytic enzyme.

### A. NBS Cleavage

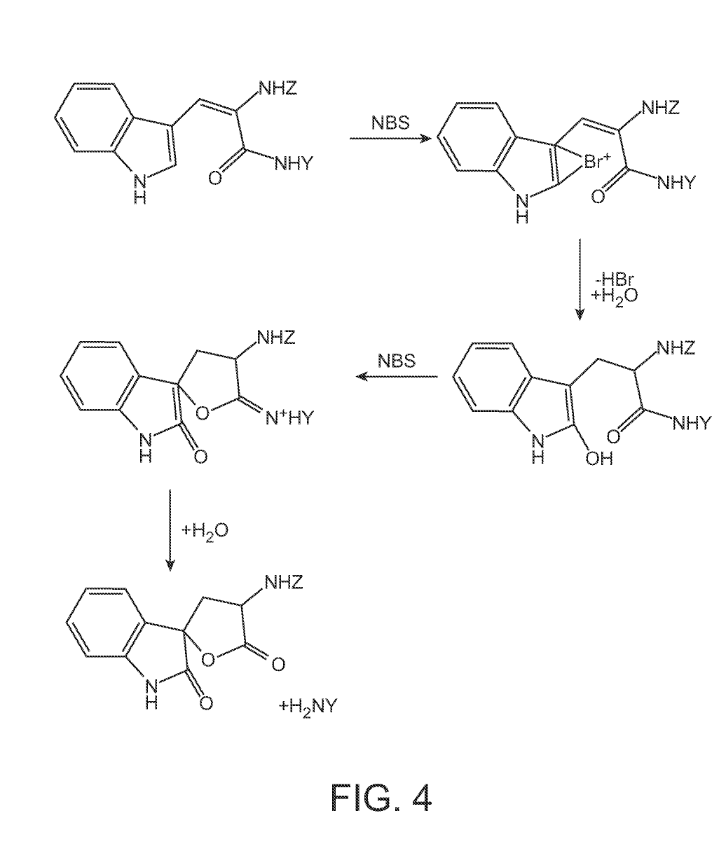

In some cases, the cleavage reaction involves the use of a mild brominating agent N-bromosuccinimde (NBS) to selectively cleave a tryptophanyl peptide bond at the amino terminus of the target peptide. Without wishing to be bound by theory, it is believed that in aqueous and acidic conditions, NBS oxidizes the exposed indole ring of the tryptophan side chain, thus initiating a chemical transformation that results in cleavage of the peptide bond at this site. FIG. 4 illustrates one possible mechanism for the selective cleavage of tryptophan peptide bonds with N-bromosuccinimde. According to the mechanism, the active bromide ion halogenates the indole ring of the tryptophan residue followed by a spontaneous dehalogenation through a series of hydrolysis reactions. These reactions may lead to the formation of an oxindole derivative which promotes the cleavage reaction.

### B. NCS Cleavage

In some cases, the cleavage reaction involves the use of a mild oxidizing agent N-chlorosuccinimde (NCS) to selectively cleave a tryptophanyl peptide bond at the amino terminus of the target peptide. Without wishing to be bound by theory, it is believed that in aqueous and acidic conditions, NCS oxidizes the exposed indole ring of the tryptophan side chain, thus initiating a chemical transformation that results in cleavage of the peptide bond at this site.

### C. Enzymatic Cleavage

In some cases, enzymes may be employed to cleave the fusion protein. For example, serine or threonine proteases that can bind to either serine or threonine, respectively, and initiate catalytic mechanisms that result in proteolysis may be used. Additional enzymes include collagenase, enterokinase factor X_{A}, thrombin, trypsin, clostripain and alasubtilisin.

### D. Additional Chemical Agents

In some cases, the cleavage agent is a chemical agent such as cyanogen bromide, palladium (II) aqua complex (such as Pd(H₂O)₄), formic acid, or hydroxylamine. For example, cyanogen bromide may be used to selectively cleave a fusion peptide at a methionine amino acid at the amino terminus of the target peptide.

### XIV. Downstream Processing

Target peptides produced according to methods described herein may be further modified. For example, the C-terminus of the target peptide can be connected to alpha-hydroxyglycine. At the desired time, the target peptide, either as the isolated target peptide or as part of the fusion peptide, can be exposed to acid catalysis to yield glycolic acid and a carboxamide group at the carboxy terminus of the target peptide. A carboxamide group at the carboxy terminus may be present in a variety of neuropeptides, and is thought to increase the half-life of various peptides in vivo.

Target peptides produced according to methods described herein may be further modified to alter in vivo activity. For example, a polyethylene glycol (PEG) group may be added to a target peptide.

### EXAMPLES

The invention is further illustrated by the following non-limiting examples.

### Example 1

### Production and Analysis of BBI Protein

This example describes the transformation of E. coli cells with an expression vector to initiate the synthesis of BBI fused to the amino acid sequence TRHK (SEQ ID NO: 3) with 1 mM IPTG (GoldBio) for the production of BBI. Plated cells were incubated overnight at 37° C. and then one colony from this plate was grown up overnight in a starter culture of 8 mL of Luria broth+kanamycin. The following morning, the starter culture was inoculated into 1 L of Luria broth+kanamycin and grown to an optical density (OD) of 2.0. At this point, the cells were induced with 1 mM IPTG (GoldBio) to initiate the synthesis of TRHK-BBI.

To optimize the amount of TRHK-BBI production in the bacteria, samples of the 1 L inoculation were taken prior to inducing the bacteria, and then 2, 4, 6, and 16 hours (overnight growth) after induction. An acrylamide gel was used to analyze the samples and select the optimal induction time.

Following induction of TRHK-BBI production in E. coli, lysis buffer containing 50 mM Tris pH 8.0, 150 mM NaCl, 1 mM EDTA, and 1% Triton X-100 was added before lysing the cells. Insoluble inclusion bodies were collected using washing and centrifugation. Three different wash buffers containing varying concentrations of Tris pH 8.0, NaCl, and Triton X100 were used. Once washed clean of the remaining cellular components, the insoluble inclusion bodies were solubilized in a buffer containing 8M urea, 0.193M Ethanoloamine and 2.5 mM DTT. The 8M urea served as a chaotropic agent in solubilizing protein.

Media collected from un-induced and induced bacteria, the cell lysate produced from high output sonication, and the supernatant from each washing step during the inclusion body preparation were run on an acrylamide gel. The gel was stained with Coomassie Blue reagent, and the appearance of a band at the appropriate molecular weight provided evidence for inclusion body synthesis resulting from induction.

The concentration of protein in solubilized inclusion bodies was determined via a Bradford Assay. A series of NCS cleavage reactions were run to determine the optimal conditions for tryptophanyl peptide bond cleavage. Three concentrations of NCS purchased from TCI America (equimolar, 3×, and 6×) were allowed to react with TRHK-BBI for varying amounts of time (0, 15, and 30 minutes) before being quenched with excess N-acetylmethionine (Sigma). Cleavage was monitored by running the cleavage product on an acrylamide gel and observing a band at the appropriate molecular weight.

SP Sepharose High Performance Chromatography resin purchased from GE was equilibrated with refolding buffer. Next, the resin was charged with refolded fusion protein and the flow through was collected. The column was then washed with five column volumes of 20 mM Tris buffer, pH8.0 to remove impurities, urea and flow through. Afterwards, NCS was loaded and flowed through the column. The column was then washed with 20 mM Tris buffer, pH7.5 to elute remaining protein of interest and the flow through was collected.

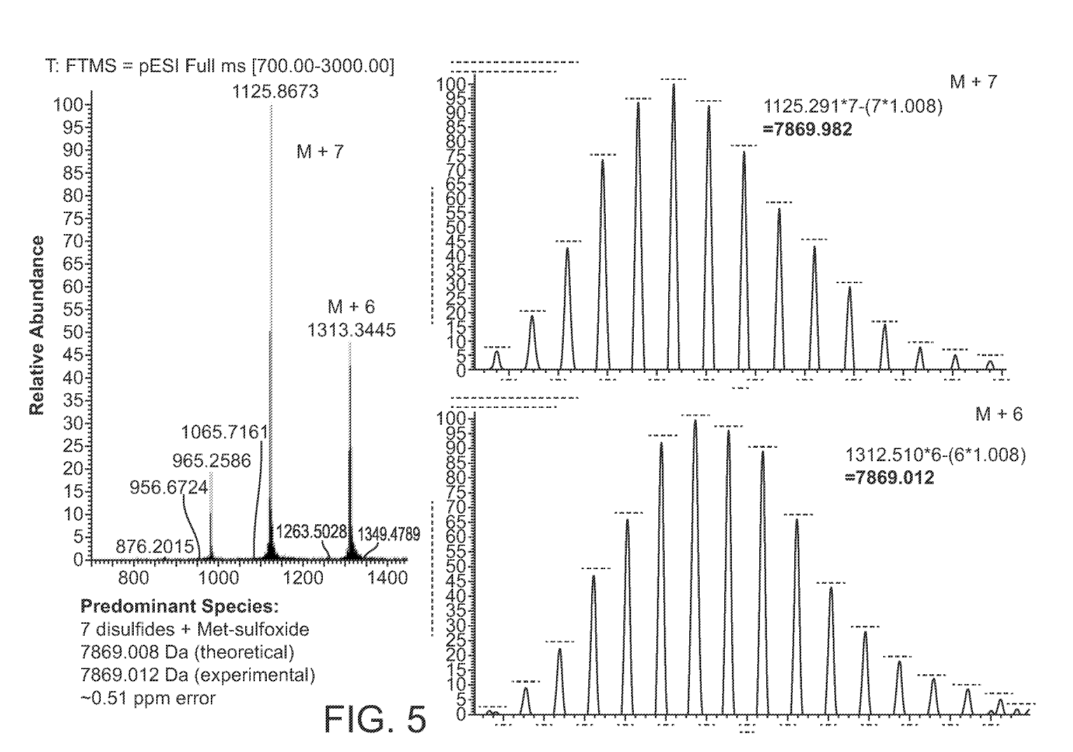

The BBI protein produced was analyzed by mass spectrometry. As shown in FIG. 5, the predominant species of BBI protein produced has a methionine sulfoxide at amino acid 27 and 7 disulfide bonds.

For the trypsin inhibition assays, several doses of (a) BBI with methionine sulfoxide at amino acid 27 (oxidized), (b) BBI without methionine sulfoxide at amino acid 27 (reduced), and (c) a mutant BBI having a M27V mutation (mutant) were prepared in reaction buffer (8.77 mL of 80 mM Tris, pH 7.8+240 μL 2M CaCl₂). A 2× stock of trypsin (6 U/mL) was prepared by first adding 3 mg of trypsin (10,000 U/mg) into 1 mL of 1 mM HCl to reach 30K U/mL. Second, a 1:3 dilution was used to prepare a solution of 10K U/mL, and third, 1:10 dilution of the 10K U/mL solution was used to prepare a solution of 1000 U/mL. 2× trypsin (6 U/mL) was prepared by adding 60 μL of 1000 U/mL trypsin into 9.94 mL of buffer. BBI was serially diluted into 2× trypsin to yield samples having 2× trypsin and 3000, 1000, 300, 100, 30, 10, 3, 1, 0.3, 0.1, 0.03, or 0 ng of each BBI. To prepare substrate, 5 mg of N-(p-Tosyl)-Gly-Pro-Lys 4-nitroanilide acetate salt was dissolved in 79 μL of 100% EtOH to reach a concentration of 100 mM. 2× substrate (0.6 mM) was prepared by adding 30 μL of 100 mM substrate to 5 mL of reaction buffer. 50 μL of each (BBI+trypsin) mixture was added into a well of a 96-well plate and mixed with 50 μL of 2× substrate to yield 1× trypsin and 1× substrate. The plate was incubated at 25° C. for 90 minutes before reading at 405 nm.

For the chymotrypsin inhibition assays, several doses of (a) BBI with methionine sulfoxide at amino acid 27, (b) BBI without methionine sulfoxide at amino acid 27, and (c) a mutant BBI having a M27V mutations were prepared in reaction buffer (8.77 mL of 80 mM Tris, pH 7.8+240 μL 2M CaCl₂) were prepared. A 2× stock of chymotrypsin was prepared by first adding 15 mg of chymotrypsin (40 U/mg) into 1 mL of H₂O to reach 600 U/mL. Second, a 1:10 dilution was used to prepare a solution of 60 U/mL. 2× chymotrypsin (0.6 U/mL) was prepared by adding 100 μL of 60 U/mL chymotrypsin into 10 mL of reaction buffer. BBI was serially diluted into 2× chymotrypsin to yield samples having 2× chymotrypsin and 300000, 100000, 30000, 10000, 3000, 1000, 300, 100, 30, 10, 3 or 0 ng of each BBI. To prepare substrate, 1 mL of DMF (N,N-Dimethylformamide) was added to 25 mg of N-Succinyl-Ala-Ala-Pro-Phe p-nitroanilide (SEQ ID NO: 12) to reach 100 mM. This is diluted 166 fold to make a 2× stock (0.6 mM). 50 μL of each (BBI+chymotrypsin) mixture was added into a well of a 96-well plate and mixed with 50 μL of 2× substrate, to yield 1× chymotrypsin and 1× substrate. The plate was incubated at 25° C. for 10 min before reading at 405 nm.

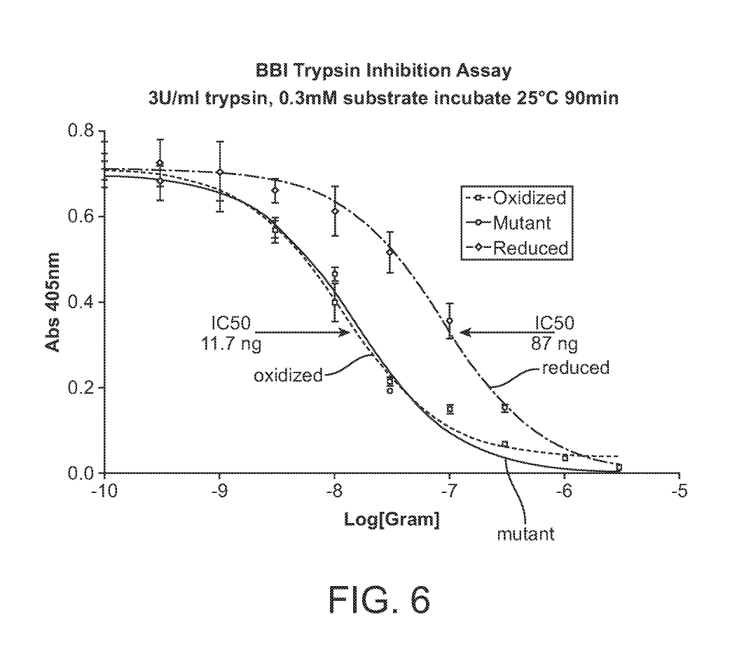

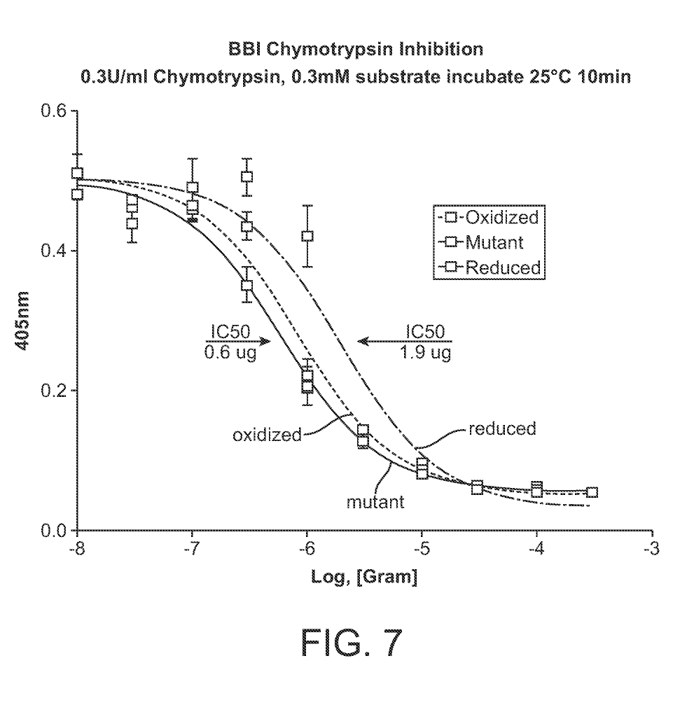

In trypsin and chymotrypsin inhibition assays, BBI protein having a methionine sulfoxide at amino acid 27 is more effective (e.g., lower IC50) compared to BBI protein not having a methionine sulfoxide at amino acid 27, as shown in FIGS. 6 and 7. In trypsin and chymotrypsin inhibitions assays, BBI protein having a methionine sulfoxide at amino acid 27 was comparable to a mutant BBI having a M27V mutation.

### Example 2

### Treatment of Stevens-Johnson Syndrome (SJS) with Soybean BBI Protein

Animal models of Stevens-Johnson syndrome (SJS) and toxic epidermal necrolysis (TEN) are generated by injecting peripheral blood mononuclear cells (PBMCs) from patients who have recovered from SJS/TEN intravenously into immunocompromised NOD/Shi-scid IL-2R≡^{null} (NOG) mice, followed by oral administration of a causative agent. After being administered a causative agent, these animal models may show conjunctival congestion and numerous cell death of conjunctival epithelium. Solutions of BBI protein having a methionine sulfoxide at amino acid 27 in saline are administered orally at dosages of 50 μg, 100 μg, 150 μg, and 200 μg per animal. Control animals are administered vehicle only. The animals are observed and clinically scored for decreased conjunctival congestion. Animals treated with BBI are expected to exhibit improvements in conjunctival congestion compared to control animals administered vehicle only.

### Example 3

### Treatment of Guillain-Barré Syndrome (GBS) Using Soybean BBI Protein

Mouse models of Guillain-Barré syndrome are generated by immunizing GM1/GD1a-deficient mice with heat-killed pylobacter jejunifter being immunized, animal models may develop Guillain-Barré syndrome-associated IgG antibodies against the GM1/GD1a sugar chain epitopes of bacterial lipo-oligosaccharides (LOS). Serum anti-GM1/GD1a antibody titers are measured by ELISA to establish a baseline. Solutions of BBI protein having a methionine sulfoxide at amino acid 27 in saline are administered orally at dosages of 50 μg, 100 μg, 150 μg, and 200 μg per animal. Control animals are administered vehicle only. Serum anti-GM1/GD1a antibody titers are measured by ELISA following treatment. The animals treated with BBI are expected to exhibit decreased levels of serum anti-GM1/GD1a antibody titers as measured by ELISA compared to control animals administered vehicle only.

### Example 4

### Treatment of Type I Diabetes Using Insulin in Combination with Soybean BBI Protein

To determine oral bioavailability of insulin administered in combination with soybean BBI protein, compositions comprising insulin peptide, soybean BBI protein having a methionine sulfoxide at amino acid 27, and a pharmaceutically acceptable excipient are administered orally in fixed dosages of soybean BBI protein and varying dosages of insulin peptide to normal, healthy animals in a test group. In a control group, animals are administered insulin peptide only at the same dosages as in the test group and without BBI protein. Following administration, insulin levels are monitored to determine oral bioavailability of insulin. Oral bioavailability of insulin administered with soybean BBI protein is expected to be greater than that of insulin administered without soybean BBI protein.

To compare the efficacy of insulin administered with soybean BBI to insulin administered without soybean BBI for the treatment of type I diabetes, animal models of type I diabetes are treated (i) in a test group with insulin+BBI and (ii) in a control group with insulin only. Animal models of type I diabetes are generated by chemical ablation of pancreatic beta cells. Following administration of either (i) insulin+BBI or (ii) insulin only, blood glucose levels are monitored to determine efficacy in regulating blood glucose levels.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A pharmaceutical composition comprising:
a soybean Bowman-Birk inhibitor (BBI) protein having at least 80% sequence identity to SEQ ID NO: 1 (DDESSKPCCDQCACTKSNPPQCRCSDMRLNSCHSACKSCICALSYPAQCFCVDI TDFCYEPCKPSEDDKEN) or a fragment thereof, and
a pharmaceutically acceptable diluent,
wherein the soybean BBI protein comprises a methionine sulfoxide, a valine, a leucine or isoleucine at amino acid 27.

2. The pharmaceutical composition of claim 1, wherein the soybean BBI protein has at least 85%, 90%, 95%, or 98% sequence identity to SEQ ID NO: 1.

3. The pharmaceutical composition of claim 2, wherein the soybean BBI protein has an amino acid sequence of SEQ ID NO: 1.

4. The pharmaceutical composition of claim 1, further comprising a peptide.

5. The pharmaceutical composition of claim 4, wherein the peptide is an insulin peptide, analogue or fragment thereof; a glucagon peptide, analogue or fragment thereof; and a glucagon-like peptide-1 (GLP-1) peptide, analogue or fragment thereof.

6. The pharmaceutical composition of any one of claims 1-4, wherein the pharmaceutical composition is formulated for oral administration.

7. A method for treating an autoimmune disease, the method comprising administering to a subject in need thereof the pharmaceutical composition of any one of claims 1-6.

8. The method of claim 7, wherein the autoimmune disease is selected from the group consisting of: type I diabetes, Stevens-Johnson Syndrome, Guillain-Barre Syndrome, anti-aquaporin 4 antibody positive neuromyelitis optica spectrum disorder, and bullous pemphigoid.

9. The method of claim 7, wherein the autoimmune disease is type I diabetes.

10. The method of claim 9, wherein the peptide is an insulin peptide, analogue or fragment thereof.

11. The method of any one of claims 7-10, wherein the pharmaceutical composition is administered orally.

12. A method for producing a target peptide, the method comprising:
(a) expressing a heterologous fusion peptide in a genetically modified cell, the heterologous fusion peptide comprising an expression tag, a cleavage tag, and the target peptide, wherein the expression tag comprises an amino acid sequence having at least 80% sequence identity to SEQ ID NO: 2 (MKAIFVLKGSLDRDPEFPSDKPHHKKHHKKHHSSGSLE) or a fragment thereof, and wherein the cleavage tag comprises a Trp (W) amino acid; and
(b) cleaving the heterologous fusion peptide to release the target peptide from the heterologous fusion peptide, thereby producing the target peptide.

13. The method of claim 12, wherein the target peptide is selected from the group consisting of: a hormone peptide, a protease inhibitor protein, and a peptide toxin.

14. The method of claim 13, wherein the target peptide is selected from the group consisting of: insulin, glucagon, glucagon-like peptide 1 (GLP-1), parathyroid hormone 1-34 (PTH-34), a single-chain relaxin-1, a single-chain relaxin-2, a single-chain relaxin-3, insulin-like peptide 3, insulin-like peptide 4, insulin-like peptide 5, insulin-like peptide 6, soybean trypsin inhibitor (STI) protein, soybean Bowman-Birk inhibitor (BBI) protein, eglin C protein, Mambalgin-1, Hg1 toxin, and Stichodactyla toxin (ShK).

15. The method of claim 14, wherein the target peptide is a soybean BBI protein having at least 80%, 85%, 90%, 95%, 98%, or 100% sequence identity to SEQ ID NO: 1 (DDESSKPCCDQCACTKSNPPQCRCSDMRLNSCHSACKSCICALSYPAQCFCVDITDFCY EPCKPSEDDKEN) or a fragment thereof.

16. The method of claim 15, wherein the soybean BBI protein comprises an oxidized amino acid.

17. The method of claim 16, wherein the soybean BBI protein comprises a methionine sulfoxide at amino acid 27.

18. The method of claim 15, wherein the soybean BBI protein comprises a valine, leucine or isoleucine at amino acid 27.

19. The method of any of claims 12-18, wherein the target peptide is at least 95% pure.

20. The method of claim 19, wherein the target peptide is at least 99% pure.

21. The method of claim 12, wherein the expression tag further comprises an affinity tag.

22. The method of claim 21, wherein the affinity tag comprises at least six amino acids having charged side chains.

23. The method of claim 21 or 22, further comprising binding the heterologous fusion peptide to an affinity material via the affinity tag.

24. The method of claim 23, wherein subsequent to binding the heterologous fusion peptide to the affinity material, the method further comprises washing the affinity material to remove unbound material.

25. The method of claim 23 or 24, wherein cleaving the heterologous fusion peptide in (b) occurs while the heterologous fusion peptide is bound to the affinity material via the affinity tag.

26. The method of claim 25, wherein the target peptide possesses a tertiary structure substantially the same as the corresponding native target peptide after cleaving.

27. The method of claim 23, wherein subsequent to binding the heterologous fusion peptide to the affinity material, the method further comprises subjecting the heterologous fusion peptide to conditions sufficient to fold the target peptide.

28. The method of claim 12, wherein the heterologous fusion peptide further comprises an inclusion-body directing peptide.

29. The method of claim 28, wherein the inclusion-body directing peptide is selected from the group consisting of: a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, and an inclusion-body directing functional homolog of BRCA2.

30. The method of claim 28 or 29, wherein prior to cleaving the heterologous fusion peptide, the method further comprises removing inclusion bodies containing the fusion peptide from the genetically modified cell and solubilizing the fusion peptide in the inclusion bodies.

31. The method of claim 12, wherein the cleaving of (b) is performed with an agent selected from the group consisting of: NBS, NCS, and Pd(H₂O)₄.

32. The method of claim 12, wherein the heterologous fusion peptide is secreted from the genetically modified cell after it is expressed.

33. The method of claim 12, further comprising lysing the genetically modified cell after the heterologous fusion peptide is expressed.

34. The method of claim 12, wherein the genetically modified cell is a bacterial cell.

35. The method of claim 34, wherein the bacterial cell is an Escherichia coli cell.

36. The method of claim 12, wherein the genetically modified cell is a yeast cell.

37. The method of claim 36, wherein the heterologous fusion peptide further comprises a secretion peptide for use in the yeast cell.

38. A vector comprising:
(a) a first nucleotide sequence encoding an expression tag;
(b) a second nucleotide sequence encoding a cleavage tag; and
(c) a third nucleotide sequence encoding a target peptide;
wherein the first, second, and third nucleotide sequences are arranged in operable combination,
wherein the expression tag comprises an amino acid sequence having at least 80% sequence identity to SEQ ID NO: 2(MKAIFVLKGSLDRDPEFPSDKPHHKKHHKKHHSSGSLE) or a fragment thereof, and wherein the cleavage tag comprises a Trp (W) amino acid.

39. The vector of claim 38, wherein the target peptide is selected from the group consisting of: a hormone peptide, a protease inhibitor, and a peptide toxin.

40. The vector of claim 39, wherein the target peptide is selected from the group consisting of: insulin, glucagon, glucagon-like peptide 1 (GLP-1), parathyroid hormone 1-34 (PTH-34), a single-chain relaxin-1, a single-chain relaxin-2, a single-chain relaxin-3, insulin-like peptide 3, insulin-like peptide 4, insulin-like peptide 5, insulin-like peptide 6, soybean trypsin inhibitor (STI) protein, soybean Bowman-Birk inhibitor (BBI) protein, eglin C protein, Mambalgin-1, Hg1 toxin, and Stichodactyla toxin (ShK).

41. The vector of claim 40, wherein the target peptide is a soybean BBI protein having at least 80%, 85%, 90%, 95%, or 98% sequence identity to SEQ ID NO: 1 (DDESSKPCCDQCACTKSNPPQCRCSDMRLNSCHSACKSCICALSYPAQCFCVDITDFCY EPCKPSEDDKEN) or a fragment thereof.

42. The vector of claim 41, wherein the soybean BBI protein has an amino acid sequence of SEQ ID NO: 1.

43. The vector of claim 38, wherein the expression tag further comprises an affinity tag.

44. The vector of claim 43, wherein the affinity tag comprises at least six amino acids having charged side chains.

45. The vector of claim 38, further comprising a nucleotide sequence encoding an inclusion-body directing peptide.

46. The vector of claim 45, wherein the inclusion-body directing peptide is selected from the group consisting of: a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, and an inclusion-body directing functional homolog of BRCA2.

47. The vector of claim 38, further comprising a nucleotide promoter sequence which is active in a bacteria cell or a yeast cell.


SEQUENCE LISTING (12 sequences)

SEQ ID NO: 1 - protein, 71 aa, Glycine sp.
DDESSKPCCDQCACTKSNPPQCRCSDMRLNSCHSACKSCICALSYPAQCFCVDITDFCYEPCKPSEDDKEN

SEQ ID NO: 2 - protein, 38 aa, Artificial Sequence
  Description of Artificial Sequence Synthetic polypeptide
MKAIFVLKGSLDRDPEFPSDKPHHKKHHKKHHSSGSLE

SEQ ID NO: 3 - protein, 4 aa, Artificial Sequence
  Description of Artificial Sequence Synthetic peptide
TRHK

SEQ ID NO: 4 - protein, 6 aa, Artificial Sequence
  Description of Artificial Sequence Synthetic peptide
HHHHHH

SEQ ID NO: 5 - protein, 10 aa, Artificial Sequence
  Description of Artificial Sequence Synthetic peptide
  MISC_FEATURE (1)..(10): This sequence may encompass 5-10 residues wherein some positions may be absent
KKKKKKKKKK

SEQ ID NO: 6 - protein, 10 aa, Artificial Sequence
  Description of Artificial Sequence Synthetic peptide
  MISC_FEATURE (1)..(10): This sequence may encompass 5-10 residues wherein some positions may be absent
HHHHHHHHHH

SEQ ID NO: 7 - protein, 10 aa, Artificial Sequence
  Description of Artificial Sequence Synthetic peptide
  MISC_FEATURE (1)..(10): This sequence may encompass 5-10 residues wherein some positions may be absent
EEEEEEEEEE

SEQ ID NO: 8 - protein, 10 aa, Artificial Sequence
  Description of Artificial Sequence Synthetic peptide
  MISC_FEATURE (1)..(10): This sequence may encompass 5-10 residues wherein some positions may be absent
RRRRRRRRRR

SEQ ID NO: 9 - protein, 6 aa, Artificial Sequence
  Description of Artificial Sequence Synthetic peptide
KKKKKK

SEQ ID NO: 10 - protein, 6 aa, Artificial Sequence
  Description of Artificial Sequence Synthetic peptide
EEEEEE

SEQ ID NO: 11 - protein, 6 aa, Artificial Sequence
  Description of Artificial Sequence Synthetic peptide
RRRRRR

SEQ ID NO: 12 - protein, 4 aa, Artificial Sequence
  Description of Artificial Sequence Synthetic peptide
AAPF